(19) United States
(12) Patent Application Publication
(10) Pub. No.: US 2009/0170144 A1
(43) Pub. Date: Jul. 2, 2009

(54) DETERMINATION OF VIABLE MICROORGANISMS USING COATED PARAMAGNETIC BEADS

(76) Inventors: William R. Heineman, Cincinnati, OH (US); Hallen Brian Halsall, Cleves, OH (US); Carl James Seliskar, Cincinnati, OH (US); Ismail Hakki Boyaci, Ankara (TR)

Correspondence Address: FROST BROWN TODD, LLC, 2200 PNC CENTER, 201 E. FIFTH STREET, CINCINNATI, OH, 45202, US

(21) Appl. No.: 11/816,797

(22) PCT Filed: Feb. 22, 2006
(86) PCT No.: PCT/US06/06186

§ 371 Date: May 30, 2008

Related U.S. Application Data
(60) Provisional application No. 60/655,204, filed on Feb. 22, 2005.

(51) Int. Cl. C12Q 1/34; C12Q 1/04; C12Q 1/00

(52) U.S. Cl. 435/18; 435/34; 435/4

(57) ABSTRACT

The present invention relates to methods for the detection of microorganisms. In one embodiment, the present invention provides methods for detecting live microorganisms in a culture by capturing and culting the microorganisms on para-tropic-coated paramagnetic beads. This technique is useful for any application in which it is necessary to monitor the biological contamination level, for example drinking water, recreational waters, food processing waters and medical laboratories. In one embodiment, the method for determining the concentration of viable microorganisms in a sample according to the invention further comprises an inducer reagent, wherein the inducer reagent includes an inducer compound that induces the activity of an enzyme unique to the microorganism of interest.

### CROSS REFERENCE TO RELATED APPLICATIONS

This application claims priority under 35 U.S.C. 119 to U.S. provisional application Ser. No. 60/655,204, filed Feb. 22, 2005; the disclosure of which is hereby expressly incorporated herein by reference in its entirety.

### FIELD OF THE INVENTION

The present invention generally relates to methods for the detection of microorganisms. In one embodiment, the present invention provides methods for detecting live microorganisms in a culture.

### BACKGROUND OF THE INVENTION

Coliforms, fecal coliforms and Escherichia coli are used as indicators of fecal contamination of water supplies and recreational waters [1]. Among these, E. coli is generally considered the most reliable since its presence directly relates to fecal contamination [2]. E. coli is found in the intestinal contents of humans, warm-blooded animals and birds. Although many strains are non-pathogenic, some strains of E. coli are involved in food and water-borne diseases [3].

The traditional methods for enumerating E. coli are time-consuming, inconvenient and in most cases, require several handling steps [4]. To overcome these difficulties, many novel, rapid methods have been developed to replace traditional techniques. In addition to being rapid, they are quite specific, sensitive, accurate and less labor intensive. Immunoassays, which rely on the specificity of the antigen-antibody reaction, are commonly used to rapidly detect pathogens. Many immunoassays, such as the commercially available enzyme-linked immunosorbent assays (ELISA) for detecting some bacteria, require a minimum of 10⁵-10⁶ cells for detection, so an enrichment step is usually incorporated to achieve a sufficient cell concentration, which increases the assay time [5]. Another option is to immunocapture the bacterial cells using magnetic immunobeads. When the beads are mixed with a sample, they capture the specific bacteria and then are removed by a magnet to concentrate the target bacteria and to remove bacteria and other components of the sample that may interfere with the analysis [5].

Like other bacteria, under nutrient deprivation and different growing conditions, E. coli undergo physiological modifications involving enzyme activities and protein synthesis [6]. Among these, β-galactosidase, a catabolic enzyme that cleaves lactose into galactose and glucose, is often used as a general marker for total coliforms. Thus, the activity of this enzyme can be used as an indicator of fecal pollution and to determine the number of bacteria using suitable substrates, in particular fluorogenic or chromogenic enzyme substrates [6]. The fluorogenic enzyme substrates generally consist of a specific substrate for the specific enzyme, such as a sugar or amino acid, and a fluorogen, such as 4-methylumbelliferone. Methylumbelliferyl-substrates are highly sensitive and very specific [7].

The present invention describes the development of a bead-based immunoassay for the detection of E. coli. The immunoassay was based on coating the surface of paramagnetic microbeads with antibody specific to E. coli and capturing the bacteria. The activity of β-galactosidase, induced in E. coli, was determined by using the substrate 4-methylumbelliferyl-β-D-galactoside (MUG) and was used to enumerate E. coli. The developed immunoassay did not require enrichment or filtration. In addition, the detection system did not require secondary antibody, because the detection was based on the activity of the intrinsic enzyme of E. coli. Thus, the activity of the enzyme was used to determine the number of live bacteria in the sample.

### SUMMARY OF THE INVENTION

The present invention relates to methods for the detection of microorganisms. In one embodiment, the present invention provides methods for detecting live microorganisms in a culture.

In general, the present invention provides a method of measuring the presence of a live microorganisms of interest in a sample, comprising the steps of:

a. capturing the microorganism of interest with an appropriate amount of targeting moiety (paratropic molecule) capable of binding specifically to the target microorganism of interest;

b. incubating the microorganism with a substrate for an enzyme present in the microorganismfor a time sufficient to allow production of a detectable amount of product by the enzyme in the live microorganisms present;

c. detecting the product; and

d. correlating the amount of product with a known standard and thereby determining the presence of live microorganisms.

The targeting moiety used is preferably antibodies, soluble receptors, paratopic molecules, recombinant molecules with binding sites for the target analyte, or fragments thereof. The targeting moiety is preferably an antibody and most preferably a polyclonal antibody which recognizes many epitopes on the target microorganism.

In another embodiment, the present invention provides a method of measuring the presence of a live microorganisms of interest in a sample, comprising the steps of:

a. capturing the microorganism of interest with an appropriate amount of targeting moiety (paratropic molecule) capable of binding specifically to the target microorganism of interest;

b. incubating the microorganism for a time sufficient to allow growth of the live microorganisms present;

c. incubating the microorganism with a substrate for an enzyme present in the microorganismfor a time sufficient to allow production of a detectable amount of product by the enzyme in the live microorganisms present;

d. detecting the amount of product produced in the sample; and

e. correlating the amount of product with a known standard and thereby determining the presence of live microorganisms.

In another embodiment, the present invention provides a method of measuring the presence of a live microorganisms of interest in a sample, comprising the steps of:

a. capturing the microorganism of interest with an appropriate amount of targeting moiety (paratropic molecule) capable of binding specifically to the target microorganism of interest;

b. incubating the microorganism for a time sufficient to allow growth of the live microorganisms present;

c. incubating the microorganism with a substrate for an enzyme present in the microorganismfor a time sufficient to allow production of a detectable amount of product by the enzyme in the live microorganisms present;

d. detecting the amount of product produced in the sample; and

e. correlating the amount of product with a known standard and thereby determining the presence of live microorganisms

f. wherein the product is detected by fluorescence.

This technique is useful for any application in which it is necessary to monitor the biological contamination level, for example drinking water, recreational waters, food processing waters and medical laboratories.

In another embodiment, the method for determining the concentration of viable coliforms or E. coli in a liquid according to the invention, further comprises the step of obtaining a sample to be tested from a source where contamination is suspected.

In another embodiment, the method for determining the concentration of viable microorganisms in a sample according to the invention further comprises an inducer reagent, wherein the inducer reagent includes an inducer compound that induces the activity of an enzyme unique to the microorganism of interest.

In one embodiment, the inducer is isopropylthiogalactopyranoside (IPTG) which is an inducer of beta-galactosidase enzyme in coliforms.

In another embodiment, the inducer is selected from the group consisting of 1-O-methyl-beta-D-glucuronide, isopropyl-beta-D-thioglucuronic acid, isopropyl-beta-D-thiogalactopyranoside, 3-O-methyl-.alpha.-D-glucopyranoside and 1-O-methyl-beta-D-glucopyranoside. Those of ordinary skill in the art will recognize that a particular inducer can by used to promote the production of a particular enzyme.

In another embodiment, the method for determining the concentration of viable coliforms or E. coli in a liquid according to the invention further comprises an indicator reagent, wherein the indicator reagent includes an indicator compound that undergoes a change detectable by spectrophotometric or visual methods upon cleavage by a beta galactosidase enzyme found in coliforms or a beta glucuronidase enzyme unique to E. coli.

In another embodiment, the indicator reagent which undergoes a visible color change when it is cleaved by enzymes unique to the coliform group of bacteria is used in the coliform test and a reagent which becomes fluorescent when it is cleaved by enzymes unique to E. coli is used in the E. coli test, wherein only viable microorganisms can cleave the reagent.

In another embodiment, the method for determining the concentration of viable microorganisms in a sample according to the invention further comprises incubating the test sample and control sample at about 35° C. for about 24 h or less.

In one embodiment, the invention is to provide a new method for rapidly and accurately detecting and indicating the presence of viable coliforms or E. coli in a liquid sample.

In another embodiment, the invention to provide a semiquantitative method for rapidly and accurately quantifying and indicating the concentration of viable coliforms or E. coli in a liquid sample.

In another embodiment, the invention to provide a method in which the detection is by use of spectrophotometry.

In another embodiment, the method for determining the concentration of viable microorganisms in a sample according to the invention further comprises lysing the cell membranes of the mircroorganism in order to release the enzyme to which the substrate is directed.

In another embodiment, the invention to provide a kit for rapidly and accurately determining and indicating the presence or absence of coliforms or E. coli in a liquid sample. Uses of the kit may be for detecting the above coliforms or E. coli, however other uses are possible. Each component of the kit(s) may be individually packaged in its own suitable container. The individual containers may also be labeled in a manner which identifies the contents. Moreover, the individually packaged components may be placed in a larger container capable of holding all desired components. Associated with the kit may be instructions which explain how to use the kit. These instructions may be written on or attached to the kit.

In one embodiment, the invention involves the binding of monoclonal antibodies, e.g. of murine or human origin, that specifically recognize antigens present on microorganism cells in question, or for other purposes to specified subpopulations of cells, to paramagnetic particles, either directly or to beads first covered with antibodies specifically recognizing the respective antibodies, or the Fc-portion of IgG antibodies, that bind to the microorganism cells. In one embodiment, the cell binding antibodies may be of the IgG or IgM type or being a fragment of ab IgG or IgM.

The present invention also provides for reagent kits useful in performing the methods disclosed, providing:

a. a first reagent containing a labeled targeting moiety specific for the target microorganism and capable of forming a complex with the target microorganism;

b. a second reagent separated from said first reagent which contains a substrate suitable for the microorganism to be detected; and

c. a third reagent separated from said first and second reagents which contains a standard for the product produced by the substrate.

The present invention also provides for reagent kits useful in performing the methods disclosed, providing:

a. a first reagent containing a labeled targeting moiety specific for the target microorganism and capable of forming a complex with the target microorganism;

b. a second reagent separated from said first reagent which contains a substrate suitable for the microorganism to be detected; and

c. a third reagent separated from said first and second reagents which contains a standard for the product produced by the substrate.

d. a fourth reagent separated from said first, second and third reagents which contains a detection label for the product.

In one embodiment, the paratropic moiety is an antibody and the capture moiety is an antibody. In another embodiment, these antibodies are polyclonal. In another embodiment, the capture antibodies are immobilized on a solid support. In another embodiment, the solid support is a microbead. In another embodiment, the microbead is a paramagnetic microbead coated with an antibody directed towards one or more microorganisms of interest.

The present invention also provides reagent kits useful in performing the disclosed methods, comprising: (a) a first container having paratopic molecules that immunoreact with a target microorgansims, and are operatively linked to an enzyme indicating means; (b) a second container having paratopic molecules that immunoreact with the target product but are not in the first container; and (c) one or more other containers comprising one or more of the following: a sample reservoir, a solid phase support, wash reagents, reagents capable of detecting presence of bound antibody from the second container, or reagents capable of amplifying the indication means.

In one embodiment, the paratopic molecules are detectably labeled through the use of a label selected from the group consisting of radioisotopes, affinity labels, enzymatic labels, and fluorescent labels. Most preferably, the paratopic molecules are detectably labeled through the use of fluorescent labeling agents are fluorochromes e.g., fluorescein isocyanate (FIC), fluorescein isothiocyanate (FITC), 5-dimethylamine-1-naphthalenesulfonyl chloride (DANSC), tetramethylrhodamine isothiocyanate (TRITC), lissamine, rhodamine 8200 or sulphonyl chloride (RB 200 SC).

In another embodiment, the present invention is directed to a method for monitoring a sample comprising measuring the concentration of a microorganism.

These and other objects, features and advantages of the present invention will become apparent after a review of the following detailed description of the disclosed embodiments and the appended claims.

### BRIEF DESCRIPTION OF THE DRAWINGS

The accompanying drawings incorporated in and forming a part of the specification illustrate several aspects of the present invention, and together with the description serve to explain the principles of the invention. In the drawings:

FIG. 1(a) Optimization of antibody concentration for coating the paramagnetic beads. (b) Effect of capture time for bead on antibody binding to form bead-antibody complex.

FIG. 2 Effect of incubation time for capturing E. coli by bead-antibody complex.

FIG. 3(a) Study of IPTG concentration for optimizing β-galactosidase activity. (b) Optimization of incubation temperature for maximizing β-galactosidase activity.

FIG. 4(a) The RDE signals generated using E. coli cultures of high & low concentrations as well as combined and individual components of the blank growth medium. These signals show the absence of significant background noise. Inset: Change of current (ΔμA) after adding the sample solutions to the RDE system. (b) PAP calibration curve generated by plotting PAP concentrations (mM) versus current (μA). PAP concentrations of 1.35×10⁻⁴ to 4.0×10⁻³ mM were used.

FIG. 5(a) Amperometric detection of PAP production in different concentrations of E. coli. Bacterial concentrations of 20 cfu/mL to 2×10⁶ cfu/mL were used. (b) Incubation time versus initial concentration of E. coli. The curve is linear with a least squares line of y=−81.8×+519.4, R²=0.989 between 20 to 2×10⁶ cfu/mL resulting from the dilution of 5 μL of the modified microbeads with 20 μL of the enzyme substrate.

In the following description of the illustrated embodiments, references are made to the accompanying drawings, which form a part hereof, and in which is shown by way of illustration various embodiments in which the invention may be practiced. It is to be understood that other embodiments may be utilized, and structural and functional changes may be made without departing from the scope of the present invention.

### DETAILED DESCRIPTION OF THE INVENTION

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention that will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All references, publications, patents, patent applications, and commercial materials mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention. In order to provide a clear and consistent understanding of the specification and claims, including the scope to be given such terms, the following definitions are provided:

“Biological activity” or “bioactivity” or “activity” or “biological function”, which are used interchangeably, for the purposes herein means a function that is directly or indirectly performed by a polypeptide (whether in its native or denatured conformation), or by any subsequence thereof.

The term “antibody” refers to a molecule that is a member of a family of proteins called immunoglobulins that can specifically combine with an antigen. Such an antibody combines with its antigen by a specific immunologic binding interaction between the antigenic determinant of the antigen and the antibody combining site of the antibody. The phrase “antibody molecule” in its various grammatical forms as used herein contemplates both an intact immunoglobulin molecule and an immunologically active portion of an immunoglobulin molecule. Exemplary antibody molecules are intact immunoglobulin molecules, substantially intact immunoglobulin molecules and those portions of an immunoglobulin molecule that contain the paratope, including those portions known in the art as Fab, Fab′ F(ab′).sub.2 and F(v). Fab and F(ab′).sub.2 portions of antibodies are prepared by the proteolytic reaction of papain and pepsin, respectively, on substantially intact antibodies by methods that are well known. See for example, U.S. Pat. No. 4,342,566 to Theofilopolous and Dixon. Fab′ antibody portions are also well known and are produced from F(ab′).sub.2 portions followed by reduction of the disulfide bonds linking the two heavy chain portions as with mercaptoethanol, and followed by alkylation of the resulting protein mercaptan with a reagent such as iodoacetamide. An antibody containing intact antibody molecules are preferred, and are utilized as illustrative herein. The phrase “monoclonal antibody.” in its various grammatical forms refers to a population of one species of antibody molecule of determined (known) antigen-specificity. A monoclonal antibody contains only one species of antibody combining site capable of immunoreacting with a particular antigen and thus typically displays a single binding affinity for that antigen. A monoclonal antibody may therefore contain a bispecific antibody molecule having two antibody combining sites, each immunospecific for a different antigen.

An “antibody combining site” is that structural portion of an antibody molecule comprised of heavy and light chain variable and hypervariable regions that specifically binds antigen. Using the nomenclature of Jerne, Ann. Immunol., 125:373-389 (1974), an antibody combining site is usually referred to herein as a “paratope.”

Antibody combining site-containing (paratope-containing) polypeptide portions of antibodies are those portions of antibody molecules that contain the paratope and bind to an antigen, and include, for example, the Fab, Fab′, F(ab′)2 and F(v) portions of the antibodies. In one embodiment, intact antibodies are used.

The word “antigen” has been used historically to designate an entity that is bound by an antibody, and also to designate the entity that induces the production of the antibody. More current usage limits the meaning of antigen to that entity bound by an antibody, whereas the word “immunogen” is used for the entity that induces antibody production. Where an entity discussed herein is both immunogenic and antigenic, it will generally be termed an antigen.

The phrase “antigenic determinant” refers to the actual structural portion of the antigen that is immunologically bound by an antibody combining site. The Jerne nomenclature redefines an antigenic determinant as an “epitope.”

“ELISA” refers to an enzyme-linked immunosorbent assay that employs an antigen or antibody bound to a solid phase and an enzyme-antibody or enzyme-antigen conjugate to detect and quantify the amount of antigen or antibody present in a sample. A description of the ELISA technique is found in in U.S. Pat. No. 3,654,090, issued Apr. 4, 1972; U.S. Pat. No. 3,850,752, issued Nov. 26, 1974; and U.S. Pat. No. 4,016,043, issued Apr. 5, 1977, all to Schuurs, et al., which are incorporated herein by reference.

“Enzyme” refers to a protein capable of accelerating or producing by catalytic action some change in a substrate for which it is often specific. “Enzyme activity” refers to a measurement of the catalytic capabilities of an enzyme to convert substrate to product usually expressed in units per weight of sample tested.

“Immunoreactant” as used herein refers to the product of an immunological reaction; i.e., that entity produced when an antigen is immunologically bound by an antibody or a molecule containing a paratope.

As used herein, the terms “label” and “indicating means” in their various grammatical forms refer to single atoms and molecules that are either directly or indirectly involved in the production of a detectable signal to indicate the presence of a complex. Any label or indicating means can be linked to or incorporated in an expressed protein, polypeptide, or antibody molecule that is part of an antibody or monoclonal antibody composition of the present invention, or used separately, and those atoms or molecules can be used alone or in conjunction with additional reagents. Such labels are themselves well-known in clinical diagnostic chemistry and constitute a part of this invention only insofar as they are utilized with otherwise novel proteins methods and/or systems.

The labeling means can be a fluorescent labeling agent that chemically binds to antibodies or antigens without denaturing them to form a fluorochrome (dye) that is a useful immunofluorescent tracer. Suitable fluorescent labeling agents are fluorochromes such as fluorescein isocyanate (FIC), fluorescein isothiocyanate (FITC), 5-dimethylamine-1-naphthalenesulfonyl chloride (DANSC), tetramethylrhodamine isothiocyanate (TRITC), lissamine, rhodamine 8200 sulphonyl chloride (RB 200 SC) and the like.

In one embodiment, the indicating group is an enzyme, such as horseradish peroxidase (HRP), glucose oxidase, or the like. In such cases where the principal indicating group is an enzyme such as HRP or glucose oxidase, additional reagents are required to visualize the fact that a receptor-ligand complex (immunoreactant) has formed. Such additional reagents for HRP include hydrogen peroxide and an oxidation dye precursor such as diaminobenzidine. An additional reagent useful with glucose oxidase is 2,2′-azino-di-(3-ethyl-benzthiazoline-G-sulfonic acid) (ABTS).

Paratopic molecules when linked to enzyme labels are also sometimes referred to herein as being enzyme-linked paratopic molecules.

The term “whole antibody” is used herein to distinguish a complete, intact molecule secreted by a cell from other, smaller, molecules that also contain the paratope necessary for biological activity in an immunoreaction with an epitope.

The paratopic molecules of the present invention can be monoclonal paratopic molecules. A “monoclonal antibody” (Mab) is an antibody produced by clones of a hybridoma that secretes but one kind of antibody molecule, and a monoclonal paratopic molecule is a monoclonal antibody or a paratope-containing polypeptide portion thereof, as is discussed below. The hybridoma cell is fused from an antibody-producing cell and a myeloma or other self-perpetuating cell line. Such antibodies were first described by Kohler and Milstein, Nature, 256, 495-497 (1975), which description is incorporated herein by reference.

The terms “monoclonal paratopic molecule” and “paratopic molecule” alone are used interchangeably and collectively herein to refer to the genus of molecules that contain a combining site of a monoclonal antibody, and include a whole monoclonal antibody, a substantially whole monoclonal antibody and an antibody binding site-containing portion of a monoclonal antibody.

As used herein, the term “biological assay conditions” is used for those conditions wherein a molecule useful in this invention such as an antibody binds to another useful molecule such as an antigen epitope. In one embodiment, this is at a pH value range of about 5 to about 9, at ionic strengths such as that of distilled water to that of about one molar sodium chloride, and at temperatures of about 4 degrees C. to about 45 degrees C.

The word “complex” as used herein refers to the product of a specific binding agent-ligand reaction. An exemplary complex is an immunoreaction product formed by an antibody-antigen reaction.

As used herein, a “targeting moiety or reagent” is a molecule that binds to a defined soluble molecular target. The targeting moiety may bind a receptor, a cytokine, a hormone, a drug, or other soluble molecule. Antibody is used throughout the specification as a prototypical example of a targeting moiety.

As used herein, a “ligand/anti-ligand pair” is a complementary/anti-complementary set of molecules that demonstrate specific binding, generally of relatively high affinity. Exemplary ligand/anti-ligand pairs include hapten/antibody, ligand/receptor, and biotin/avidin. Biotin/avidin is used throughout the specification as a prototypical example of a ligand/anti-ligand pair.

As defined herein, an “anti-ligand” demonstrates high affinity, bivalent or univalent binding of the complementary ligand. Preferably, the anti-ligand is large enough to avoid rapid renal clearance, and has an in vivo half-life greater than the ligand The anti-ligand should not cause the production of large ligand/anti-ligand aggregates which could be removed rapidly from blood or lymph by the reticulo-endothelial system.

As defined herein, “avidin” includes avidin, streptavidin and derivatives and analogs thereof that are capable of high affinity, multivalent or univalent binding of biotin. As defined herein, a “ligand” is a relatively small, soluble molecule that exhibits rapid serum, blood and/or whole body clearance when administered intravenously in an animal or human.

Coliform bacteria are indicators of the sanitary quality of water and food. Total coliforms (TC) in water originate from soil or organic vegetal material. Faecal (thermotolerant) coliforms (FC) and E. coli in particular inhabit the intestine of humans and animals and are indicators of faecal pollution.

Traditional processes for detecting coliforms and E. coli by membrane filtration are based on lactose fermentation in conjunction with confirmatory tests and require 48 to 96 hours to complete. A procedure is conventionally considered to be rapid if it takes 24 hours or less to perform. However, a 24 hours method is still not rapid enough to be used for the analysis of drinking water in emergency situations, e.g. after breakdowns in the water supply or construction works to the distribution system. In those cases, the detection of at least 1 coliform bacterium per 100 ml of water should be feasible within the ordinary work shift of 8 hours and preferably in maximum 7 hours to demonstrate the potability of the water and, hence to avoid unnecessary warnings to the public about the contrary.

Existing rapid (24 hours) membrane filtration methods for the detection of coliform bacteria, in particular TC and E. coli rely on the demonstration of the activity of 2 specific marker enzymes in the bacterial colonies, i.e. -galactosidase and -glucuronidase, respectively, which the bacteria produce as they grow and metabolize. The presence of these enzymes is revealed by the ability of the bacteria present on the membrane filter to cleave chromogenic substrates added to the growth medium such as 5-bromo-4-chloro-3-indolyl-D-galactopyranoside (X-gal) for -galactosidase and 5bromo-4-chloro-3-indolyl-D-glucuronide (X-gluc) for -glucuronidase. The chromogenic substrates themselves are not colored so that the detection of colored colonies on the membrane filter indicates the presence of the enzyme and, hence, of the bacteria. See e.g. Manafi and Kneifel, Zentralbl. Hyg. 189:225-234 (1989), Brenner et al., Appl. Environ. Microbiol. 59:3534-3544 (1993) and Frampton and Restaino, J. Appl. Bacteriol. 74:223-233 (1993).

Similarly, fluorogenic substrates, e.g. 4-methylumbelliferyl-D-galactopyranoside (MU-gal) or 4-methylumbelliferyl-D-glucuronide (MUG) added to the growth medium can be cleaved by bacterial -galactosidase and -glucuronidase, respectively, to yield a fluorescent product, 4-methylumbelliferone (4-MU). The fluorogenic substrates themselves do not fluoresce so that the detection of fluorescent colonies on the membrane filter indicates the presence of the enzyme and, hence, of the bacteria. Currently, the most rapid fluorescent method to detect TC on a membrane filter using MU-gal as a substrate for -galactosidase takes 16-24 hours to complete (Brenner, cited above). For E. coli the minimal detection time obtained by using MUG as a substrate for -glucuronidase is 7.5 hours (Sarhan and Foster, J. Appl. Bacterial. 70:394-400 1991)). The Berg et al. U.S. patent and scientific publication disclose a method to detect faecal (thermotolerant) coliforms on a membrane filter using an agar growth medium containing MU-gal as a substrate for -galactosidase and an incubation temperature of 41.5° C., in a time period of 6 hours (Berg et al., U.S. Pat. No. 5,292,644 and Appl. Environ. Microbiol. 54:2118-2122 (1988)). However, the time to detect total coliforms which grow at 35°-37° C. and possess lower -galactosidase activity than the thermotolerant coliforms exceeds 8 hours. E. coli cannot be detected specifically using this method.

In one embodiment, the present invention provides for a technique for rapidly and accurately detecting microorganism contamination in a liquid has been discovered. This technique is useful for any application in which it is necessary to monitor the biological contamination level, for example drinking water, recreational waters, food processing waters and medical laboratories. The water sample is added to a reagent mixture containing a chromogenic agent which yields a yellow chromophore upon cleavage by the beta galactosidase enzyme unique to the coliform group of bacteria or a reagent mixture containing a fluorogenic agent which yields a bright blue fluorophore upon cleavage by the beta glucuronidase enzyme unique to E. coli.

The nutrient formulation includes a buffer, such as phosphate buffer, capable of maintaining the pH of the sample at or near pH 7, tryptic soy broth (TSB) without glucose, succinate, and isopropylthiogalactopyranoside (IPTG) which is an inducer of beta galactosidase enzyme in coliforms. TSB is a nondefined mixture component which provides vitamins, minerals and trace elements, but no significant carbon source other than amino acids. TSB without glucose can be used by many microbes as well as the target microorganisms of the present invention for growth. Antibiotics are optionally excluded from the nutrient formulation.

Succinate is a carbohydrate source for growing organisms and is used to increase biomass. It does not inhibit production or activity of the beta galactosidase enzyme in the coliform assay but does inhibit production/activity of the glucuronidase enzyme of E. coli. Therefore, succinate is not included in the reagent powder mixture for the E. coli assay. Succinate is used in an amount effective to enhance biomass formation in the coliform assay and is usually 0.05-0.2/ml, preferably 0.1-0.15 mg/ml of sample at which concentration the biomass is rapidly increased. The addition of increased amounts of sodium succinate, for example, 0.2 mg/ml of sample, results in increased biomass of non-target microbes able to use succinate as a carbon source.

The concentration of TSB without glucose in the test ampoule after adding the sample directly without dilution should be sufficient to provide the nutrients to sustain the viability and reproduction of the target microbes, and is usually 5-15 mg/ml of sample, preferably 8-12 mg/ml, more preferably 9-11 mg/ml and most preferably 10 mg of TSB/ml of sample after direct addition of the sample to the test ampoule. The total amount of the TSB without glucose, buffer, IPTG and succinate is sufficient to sustain the viability of the target microbe (coliform) and to result in replication of the target microbe to generate sufficient biomass to produce a detectable change in the sample due to beta-galactosidase activity and is usually in the range of 10-25 mg/ml, preferably 10-20 mg/ml, more preferably 13-18 mg/ml, and most preferably 16.8-17.3 mg/ml. For example, a mixture of TSB/buffer and succinate in a ratio of 10 mg:7 mg:0.1 mg respectively is delivered in a weight of 136.8 mg for a sample of 8 ml or 171 mg for a sample of 10 ml or 342 mg for a sample of 20 ml. IPTG is used in an amount to induce the production of beta-galactosidase, and is usually in the range of 0.01-0.05 mg/ml, preferably 0.015-0.05 mg/ml, and most preferably 0.02 mg/ml of sample.

ONPG is used in an amount sufficient to produce a spectrophotometrically or visually detectable change in response to being cleaved by beta-galactosidase enzymes, and is usually in the range of 0.5-5 mg/ml, preferably 1-3 mg/ml, and most preferably 1.25 mg/ml of sample.

MUG is used in an amount sufficient to produce a spectrophotometrically or visually detectable change in response to being cleaved by beta-glucuronidase enzyme, and is usually in the range of 0.005-0.5 mg/ml, preferably about 0.05 mg/ml of sample.

The buffer may be any buffer which is used in a sufficient quantity to maintain the pH of the sample to be tested at about 7. Preferably, the buffer is a mixture of NaH2 PO4 and Na2 HPO4, and is usually in the range of 5-9 mg/ml, preferably 6.5-7.6 mg/ml, and most preferably 7 mg/ml of sample.

The sample is mixed and incubated at a temperature which allows rapid growth of the microorganism(s) being assayed and is usually 32-37° C., preferably at or near 35° C. The absorbance spectrum of each coliform test sample is monitored at or near the lambda max of the chromophore generated (at 405 nm, the lambda max of the nitrophenol chromophore generated by cleavage of the indicator reagent, ONPG, by the beta galactosidase enzyme in the coliform test, and at 355 nm, the lambda max of the fluorophore produced by cleavage of the indicator reagent, MUG, by the beta glucuronidase enzyme, in an E. coli test).

Spectrophotometric monitoring of the reaction mixture results in detection of a positive endpoint (i.e. increase in Absorbance of about 0.05 absorbance units) earlier than is possible for visual detection of the bright yellow color or detection of the bright blue fluorescence under long wave UV. Detection by visual or spectrophotometric methods can easily be accomplished within about 24 hours or less. The concentration of coliforms in the sample can be determined over a large concentration range, with spectrophotometric detection of 20 coliforms/ml within about 10 h, and visual detection within about 11.5 h. The concentration of E. coli in the sample can be determined over a large concentration range, with the detection of 10 E. coli/ml within about 12 h, preferably within 10 h using the spectrophotometric assay, and within 12 h for visual detection under long wave

In one embodiment of the present invention, the inducer is selected from the group comprising isopropyl-D-thiogalactopyranoside for -galactosidase and isopropyl-D-thioglucuronide and pnitrophenyl-D-alucuronide for -glucuronidase.

In another embodiment of the present invention, use is made as the growth medium of a medium containing mineral nutrients, a protein hydrolysate, in particular tryptone, and a sugar, preferably maltose, or a polyalcohol, preferably mannitol.

The use of such a growth medium in the preincubation step combines the properties of efficient growth promotion, good recovery of stressed coliforms/E. coli on one hand with a low luminescent background and minimal effects of quenching of light emission on the other hand.

In a still further preferred embodiment of the present invention, use is made of fluorogenic substrates different from the above mentioned MU-gal and MUG, in particular of 4-trifluoromethylumbelliferyl-D-galactopyranoside (TFMU-gal) or 4-trifluoromethylumbelliferyl-D-glucuronide (TFMUG), but preference is given to chemiluminogenic substrates. The latter have not been applied so far for the detection of bacterial colonies grown on a membrane filter but yield more sensitivity than the presently used substrates. The term total coliforms (TC) refers to bacteria belonging to either of four genera, i.e. Escherichia, Enterobacter, Klebsiella or Citrobacter, and possessing the enzyme -galactosidase. The term faecal coliforms (FC) refers to (thermotolerant) bacteria belonging to the group of the coliforms and inhabiting the intestine of humans and animals. These faecal coliforms are indicators of faecal pollution and posses also the enzyme -galactosidase, the particular species E. coli possessing further the enzyme -glucuronidase. Detection of the faecal coliforms can be done by incubating them at a higher temperature (41.5°-44° C.) than the temperature used for detecting total coliforms (about 35°-37° C.).

The term preincubation refers to a step in the method of this invention in which a sample with one or more bacteria is placed on a growth medium and kept at a certain temperature for a given time in order to propagate the bacteria and to induce the marker enzyme.

The term membrane permeabilizer refers to any compound capable of disrupting both the outer and the cytoplasmic membrane of bacteria so as to facilitate the uptake of chemicals.

The term enzyme assay refers to a step distinct from the growth step in the method of this invention in which a substrate is cleaved by a marker enzyme, in particular -galactosidase or -glucuronidase, present in the bacteria, the cleavage product then being determined by virtue of the light it emits after photochemical or chemical excitation.

The term luminescence refers to fluorescence or chemiluminescence. The term fluorescence refers to a physicochemical process in which a molecule emits light of a certain wavelength after photochemical excitation, i.e. with light of a shorter wavelength. The term chemiluminescence refers to a physicochemical process in which a molecule emits light after chemical excitation with a formulation termed “accelerator”. The term fluorogenic substrate refers to a compound which itself is non-fluorescent but which contains a structural part, i.e. the so-called fluorescent product, that does emit light when liberated from the parent compound and photochemically excited. The term chemiluminogenic substrate refers to a compound which itself is not chemiluminescent but which contains a structural part, i.e. the chemiluminescent product, that does emit light when liberated from the parent compound and chemically excited.

The sample to be analyzed is liquid or liquefied and is suspected of containing at least 1 TC, 1 FC or 1 E. coli/100 ml. Typical samples to which the method of the invention can be applied include drinking water, bathing water or liquid extracts of foods or pharmaceuticals.

In one embodiment, the assay medium contains in particular a fluorogenic substrate for either of the two marker enzymes, that is preferably TFMU-gal (.lambda.exc 394, .lambda.em 489 nm) (-galactosidase) or TFMUG (.lambda.exc 394, .lambda.em 489 nm) (-glucuronidase). The common fluorogenic substrates MU-Gal (-galactosidase) or MUG (-glucuronidase) can also be used but yield a lower sensitivity. A disadvantage of the latter two compounds is that they require spraying of the membrane filter with sodium hydroxide to yield optimal fluorescence. Other analogues of MU-gal that could also be considered as substrates for -galactosidase, including 3-acetyl-7-(-D-galactopyranosyloxy)coumarin (.lambda.exc 420, .lambda.em 459 nm), 3-(2-benzoxazolyl)-7-(-D-galactopyranosyloxy)coumarin and 1-(-D-galactopyranosyloxy)-pyrene-3,6,8-tris-(dimethyl-sulfonamide) (.lambda.exc. 495 nm, .lambda.em. 550 nm at pH 9) (see Koller et al., Appl. Fluoresc. Technol. 1, 15-16 (1989)) are in principle more sensitive and specific than MU-gal itself as their wavelengths of excitation and emission have shifted to higher values and/or because they have increased molar absorption coefficients. Non-umbelliferyl fluorogenic galactopyranosides and glucuronides can in principle also be used, for example fluorescein di-D-galactopyranoside or fluorescein-di-D-glucuronide and derivatives thereof such as C12-fluorescein-di-D-galactopyranoside or C12-fluorescein-di-D-glucuronide (ImaGene Green, Molecular Probes, Eugene, Ore.) or resorufin-D-galactopyranoside or resorufin-D-glucuronide (ImaGene Red, Molecular Probes). A still higher intrinsic sensitivity is associated with the chemiluminogenic AMPGD (3-(4-methoxyspiro>(1,2-dioxetane-3,2′-tricyclo>3.3. 1.1.sup.3.7 !decan!-4-yl)phenyl)-D-galactopyranoside) or derivatives thereof, in particular chloro derivatives (for example Galacton.™. (a mono-chloro derivative of AMPGD), Galacton-Plus.™. (a di-chloro derivative of AMPGD) (-galactosidase) available from Tropix, Inc., Bedford, Mass.) and Glucuron.™. (3-(4-methoxyspiro>1,2-dioxetane-3,2′-(5′-chloro)-tricyclo>3.3.1.1.sup.3.7 !decan!4-yl)phenyl)-D-glucuronide) or derivatives thereof (-glucuronidase) (Tropix Inc.), chemiluminescence in general being superior in sensitivity to fluorescence by the order of magnitude. AMPGD and Glucuron have been used as substrates in gene reporter assays. See e.g. Jain et al., Anal. Biochem. 199:119-124 (1991) and Bronstein et al., Anal. Biochem. 219:169-181 (1994). In another embodiment, the assay medium will contain a membrane permeabilizer. In one embodiment, the permeabilizer is polymyxin B sulfate or colistin methanesulfonate, or a mixture of one of these with lysozyme, and buffering substances to adjusted to a pH 7-7.5, in one embodiment, a pH 7.3.

In another embodiment of this invention, it is possible to include an inducer in with the liquid sample comprising bacteria. The inducer is specific for one or more proteins such as one or more enzymes in a bacteria and enhances the level of transcription and therefore the amount of protein (e.g., enzyme) in the bacteria. A variety of inducers are known in the art for a variety of enzymes. Exemplary inducers include, but are not limited to, 1-O-methyl-beta-D-glucuronide or isopropyl-beta-D-thioglucoronic acid for beta-glucuronidase enzyme activity, isopropyl-beta-D-thiogalactopyranoside for beta-galactosidase enzyme activity, 3-O-methyl-alpha-D-glucopyranoside for alpha-glucosidase enzyme activity, and 1-O-methyl-beta-D-glucopyranoside for beta-glucosidase enzyme activity.

After incubation of the cell sample with the primary antibodies or antibody fragments and the antibody-coated paramagnetic particles as described in previously, the cell suspension is incubated with a second set of antibodies or antibody fragments directed against other extracellular or against intracellular determinants of the target microorganism cells, with or without pretreatment with cell fixatives such as formaldehyde or alcohols. These antibodies or their fragments may have been prelabeled by fluorescent agents, metallocolloids, radioisotopes, biotin-complexes or enzymes like peroxidase and alkaline phosphatase, allowing visualization by per se known methods in the microscope and/or a suitable counting device.

The target microorganism cells will both be visualized with the latter method and have bound particles to their surface, and can thus be enumerated.

For use in the new procedure, in one embodiment the kits will contain for example precoated paramagnetic particles prepared for each monoclonal antibody. In another embodiment the kits contain paramagnetic particles precoated with IgG isotype specific anti-mouse or anti-human antibody as one part of it, and different target cell-associated, for example E. coli cells, antibodies as another part. In a third embodiment the kit contains paramagnetic particles precoated with specific anti-Fc antibodies, such as polyclonal anti-mouse, or monoclonal rat anti-mouse, or anti-mouse, or anti-human antibodies, capable of binding to the Fc-portion the target-cell associating antibodies, bound to specific anti-target-cell antibodies. In a further embodiment the kit contains other specific antibodies or antibody fragments directed against antigens/receptors within or on the wanted target-cells, where said antibodies or antibody fragments are conjugated to peroxidase, alkaline phosphatase, or other enzymes, together with relevant substrates to such enzymes, or where said antibody or antibody fragment is bound to non-paramagnetic particles with specific colours or with bound enzymes such as peroxidase and alkaline phosphatase.

The term “solid phase support” means any support capable of binding antigen or antibodies. Well-known supports, or carriers, include, but are not limited to, polystyrene, polypropylene, polyethylene, glass, dextran, nylon, amylases, natural and modified celluloses, polyacrylamides, agaroses, and magnetite. The support material may have virtually any structural configuration so long as on its surface, the antigen is capable of binding to an antibody. Thus, the support configuration may be spherical, as in a bead, or cylindrical, as in the inside surface of a test tube, or the external surface of a rod. Alternatively, the surface may be flat such as a sheet, test strip, etc. A preferred carrier is the bottom and sides of a polystyrene microtiter plate well. Those skilled in the art will know many other suitable carriers for binding antibody or antigen, or will be able to ascertain the same by use of routine experimentation.

The binding activity of a given lot of paratropic molecule may be determined according to well known methods. Those skilled in the art will be able to determine operative and optimal assay conditions for each determination by employing routine experimentation.

Other such steps as washing, stirring, shaking, filtering and the like may be added to the assays as is customary or necessary for the particular situation.

Detection of the labeled antibody or binding partner for the labeled analyte may be accomplished by a scintillation counter, for example, if the detectable label is a radioactive gamma emitter, or by a fluorometer, for example, if the label is a fluorescent material. In the case of an enzyme label and a chromogenic substrate, the detection can be accomplished by colorimetric methods. Detection may also be accomplished by visual comparison of the extent of enzymatic reaction of a substrate in comparison with similarly prepared standards.

Additionally, immunoassays rely on epitopes for recognition of the targeted agent. Epitopes, however, are vulnerable to modifications that can occur as a result of various constituents present in the water, such as, for example, disinfection agents (i.e., chlorine, chloramines, chlorine dioxide, hypochlorite, ozone), and residuals thereof. For example, oxidation due to the aquated chlorine or chloramines used for disinfection and maintained at residual levels in finished water can cause epitope alteration. As a further example, the amino acid side chains of tyrosine, tryptophan, cysteine, proline and histidine can also be modified by the addition of various disinfection agents. As a result, the alterations may render epitopes and/or nucleic acid sequences non-reactive towards the molecular recognition elements that have been developed for the unmodified version of the targeted agents.

As a further example, the presence of metal ions in a testing sample, such as calcium, magnesium, as well as other metals known in the art can react with the antibody or fragment (molecular recognition element) and/or the targeted agent. The interference can be caused, for example, by the metal ions present coordinating with, for example, amine, sulfhydryl, histidyl, and/or carboxyl surface ligands. This interference, however, can be circumvented, for example, by adding a chelating agent that associates with the metal ions and renders them unable to interact with the molecular recognition element and/or the targeted agent. The term “chelating agent” as used herein refers to any organic or inorganic compound that will bind to a metal ion having a valence greater than one. A “chelator”, “chelating resin”, “binder”, “sequestration agent”, or “sequester of divalant cations” refers to a composition that binds divalent cations. The binding can be reversible or irreversible. Binding of the divalent cations generally renders them substantially unable to participate in chemical reactions with other moieties with which they come in contact. A “chelator”, “chelating resin”, “binder”, “sequestration agent”, or “sequester of divalant cations” refers to a composition that binds divalent cations. The binding can be reversible or irreversible. Binding of the divalent cations generally renders them substantially unable to participate in chemical reactions with other moieties with which they come in contact. Examples of chelating agents are, for example, ethylenediaminetetraacetic acid (EDTA), nitriloacetic acid (NTA), diethylenetriaminepentaacetic acid (DTPA), trans-1,2-diaminocyclohexanetetraacetic acid (DCTA), bis-(aminoethyl)glycoether-N,N,N′N′-tetraacetic acid (ECTA), triethylene tetramine dihydrochloride (TRIEN), ethylene glycol-bis (beta.-aminoethyl ether)-N,N,N′,N′-tetracetic acid (EGTA), triethylenetetramine hexaacetic acid (TTG), deferoxamine, Dimercaprol, edetate calcium disodium, zinc citrate, penicilamine succimer, editronate as well as others known in the art. In one embodiment of the present invention the chelating agent has a concentration in the solution of between about 0.1 mM and about 50 mM. In another embodiment, the concentration of the chelating agent is between about 0.1 mM and about 10 mM. In another embodiment of the present invention, the chelator is provided in an amount such that the chelator comprises about 0.001M to about 0.05M, in one embodiment from about 0.005M to about 0.02M, and in another embodiment from about 0.008M to about 0.012M of the final chelator/finished water/(optional) buffer solution.

The chelator can be combined with finished water sample before, during, or after addition of the buffer mixture or acid to the finished water. Thus, for example, the chelator can be provided in the storage and/or preservation fluid provided with a finished water collection device. The chelator is then combined with the finished water during storage and transport. Alternatively, the chelator can be combined with the finished water just before application of the finished water sample to the assay device. In yet another embodiment, the chelator can be added to the assay device after application of the finished water or it can be stored in a reservoir within the assay device. In another embodiment, the chelator need not be combined, but only contacted with the finished water and/or the finished water/buffer mixture. Thus, for example where the immunoassay involves progression of the fluid through a porous matrix, the matrix material itself can be made of a material that chelates or otherwise sequesters or binds to divalent cations. Such matrix materials are well known to those of skill in the art. The most common sequestration agents are often used as ion exchange resins and include, but are not limited to chelex resins containing iminodiacetate ions, or resins containing free base polyamines, or amino-phosphonic acid. Alternatively, the finished water or finished water/buffer mixture can be pretreated by passage through a matrix that chelates or otherwise sequesters divalent cations. This pretreatment can be incorporated into the storage and transport container, provided as filtration step, or provided as a component of the method of extraction of the finished water sample from the collection device. In this latter embodiment, for example, centrifugation of the finished water sample out of the collection device can entail passage of the finished water through a chelation or sequestration matrix in route to a collection chamber which may or may not itself be provided as a component of the immunoassay device.

Additionally, pH levels in the testing solution can also interfere with molecular recognition due to its effect on the protonation state of acidic and basic groups on the surface of either the molecular recognition element and/or the targeted agent. Such chemical moieties that can be affected by pH include for example, histidine, carboxylic acid, amines, as well as others known in the art. This interference can be avoided, for example, by adding a buffering agent. Buffering agents are compounds whose solutions act to resist changes in pH from the addition of base or acid. The term “pH buffering agent” as used herein refers to any organic or inorganic compound or combination of compounds that will maintain the pH of a finished water sample or solution to within about 0.5 pH units of a selected pH value. A typical buffer consists of a weak acid and its conjugate base, and is chosen to operate in a particular pH range, or for other properties important to the buffered system. For example, phosphate buffers are commonly used to buffer solutions of phosphatase enzymes because they inhibit the catalytic properties of the enzymes. A pH buffering agent may be selected from, but is not limited to, Tris (hydroxymethyl) aminomethane (tromethaprim; TRIZMA base), or salts thereof, sodium and/or potassium phosphate, 2-(N-Morpholino)ethanesulfonic acid, 3-(N-Morpholino)propanesulfonic acid, N-2-Hydroxyethylpiperazine-N′-2-ethanesulfonic acid, Tris(hydroxymethyl)aminomethane, as well as phosphates or any other buffering agent that is physiologically acceptable in finished water. In one embodiment, the pH buffering agent is Tris (hydroxymethyl) aminomethane (TRIZMA Base), has a concentration in the antimicrobial solution of between about 10 mM and about 100 mM, and maintains the pH in the range of about 6.0 to about 9.0. While one of ordinary skill in the art will recognize that any physiologically acceptable concentration and pH value is within the scope of the present invention, in another embodiment the buffering agent is 50 mM Tris and maintains the pH value at about 7.0 to about 8.0.

A reducing agent can also be used. In one embodiment, the reducing agent is selected from the group consisting of dithiothreitol (DTT), thioglycerol, and mercaptoethanol. In one embodiment, the concentration of reducing agent is from about 1 mM to about 200 mM. In one embodiment, the buffering agent is sodium phosphate or sodium borate, at pH 6.5, is from about 15 mM to about 100 mM. In another embodiment, the chelating agent is ethylenediaminetetraacetic acid (EDTA). Generally, the concentration of EDTA is from about 1 mM to about 10 mM. In another embodiment, the detergent is sodium dodecyl sulfate (SDS) or polyoxyethylenesorbitan monolaurate. In one embodiment, the concentration of detergent is from about 0.01% to about 0.5%.

Carriers can also be added to the testing sample. The term “carrier” as used herein refers to any pharmaceutically acceptable solvent of chemicals, chelating agents and pH buffering agents that will allow the composition of the present invention to be added to the finished water. A carrier as used herein, therefore, refers to such solvent as, but is not limited to, water, saline, physiological saline, ointments, creams, oil-water emulsions or any other solvent or combination of solvents and compounds known to one of skill in the art that is pharmaceutically and physiologically acceptable in finished water.

Additionally diluents can be added. Where a diluent is provided, suitable diluents are chosen to be compatible with the analyte and with the target antibodies and/or proteins in the subject assay. Typically the diluents are chosen to avoid denaturation or other degradation of the proteins or antibodies and to provide a milieu compatible with and facilitating of antibody/target (epitope) binding. While any diluent typically used in immunoassays is suitable (See, e.g., Current Protocols in Immunology Wiley/Greene, NY; Harlow and Lane (1989); Antibodies: A Laboratory Manual, Cold Spring Harbor Press, NY; Stites et al. (eds.) Basic and Clinical Immunology (4th ed.) Lange Medical Publications, Los Altos, Calif., and references cited therein), a particularly embodiment diluent comprises 0.1M NaHCO3, pH 8.0. A preservative can also be added (e.g., about 0.01% thimerosal). In one embodiment, diluents are buffers ranging from about pH 7 to about pH 9, in another embodiment, from about pH 7.5 to about pH 8.5, and in another embodiment around pH 8. One of skill in the art will appreciate that the diluent (sample buffer) can additionally include a protein or other moiety unrelated to the analyte which participates in non-specific binding reactions with the various components of the assay (e.g., the substrate) and thereby blocks and prevents non-specific binding of the antibodies. In one embodiment, the blocking agent is bovine serum albumin (BSA) or polyvinyl alcohol (PVA). In one embodiment, the finished water sample is diluted at a diluent:sample ratio ranging from about 1:1 up to about 1:20 (v/v), in another embodiment from about 1:1 up to about 1:15 (v/v) and in yet another embodiment from about 1:1 up to about 1:10 (v/v). In one embodiment, the sample is diluted at a diluent:sample ratio of about 1:8 (v/v). In certain embodiments, the finished water sample may not be diluted at all prior to use.

In another embodiment, the blocking agent of non-specific binding is gelatin or bovine serum albumin. Generally, the blocking agent of non-specific binding is gelatin. In one embodiment, the concentration of gelatin is from 0.05% to about 1.0%. In another embodiment, the chaotropic agent is sodium thiocyanate or ammonium thiocyanate. In another embodiment, the antigen diluent or buffer comprises 25 mM sodium phosphate, pH 6.5, 5 mM EDTA, 10 mM DTT, 0.2% gelatin, 100 mM ammonium thiocyanate, 0.09% sodium azide and 0.1% SDS.

Bacteria are small, single-celled organisms that can generally be grown on solid or in liquid culture media. Most bacteria do not cause illness in human, but those that do generally cause illness by either invading tissue or producing poisons or toxins. Bacteria that can be harmful to humans are, for example, Brucella sp., Escherichia coli (O157:H7), Francisella tularensis, Vibrio cholerae, Corynebacterium diphtheriae, Burkholderia mallei, Burkholderia pseudomallei, Yersinia pestis, Salmonella typhosa, Bacillus anthrascis, Aerobacter aerogenes, Aeromonas hydrophila, Bacillus cereus, Bacillus subtilis, Bordetella pertussis, Borrelia burgdorferi, Campylobacter fetus, C. jejuni, Corynebacterium diphtheriae, C. bovis, Cytophagia, Desulfovibrio desulfurica, Edwardsiella, enteropathogenic E. coli, Enterotoxin-producing E coli, Flavobacterium spp., Flexibacter, Helicobacter pylori, Klebsiella pneumoniae, Legionella pneumophiia, Leptospira interrogans, Mycobacterium tuberculosis, M. bovis, N. meningitidis, Proteus mirabilis, P. vulgaris, Pseudomonas aeruginosa, Rhodococcus equi, Salmonella choleraesuis, S. enteridis, S. typhimurium, S. typhosa, Shigella sonnet, S. dysenterae, Staphylococcus aureus, Staph. epidermidis, Streptococcus anginosus, S. mutans, Vibrio cholerae, Yersinia pestis, Y. pseudotuberculosis, Actinomycetes spp., Streptomyces reubrireticuli, Streptoverticillium reticulum, and Thermoactinomyces vulgaris as well as others known in the art.

Under special circumstances, some types of bacteria form endospores that are more resistant to cold, heat, drying, chemicals, and radiation than the bacterium itself. Examples of such spores that can be harmful to humans as a source of the bacterium are, for example, Bacillus anthracis, Clostridium botulinum, as well as others known in the art.

Viruses are the simplest type of microorganism and consist of a nucleocapsid protein coat containing genetic material, i.e., DNA or RNA. Because viruses lack a system for their own metabolism, they require living hosts for replication. Most viruses do not respond to antibiotics. Viruses that can be harmful to humans are, for example, the Marburg virus, Junin virus, Rift Valley Fever virus, variola virus, Venezuelan equine encephalitis virus, yellow fever virus, Dengue viruses (DEN-1, DEN-2, DEN-3 and DEN-4), Ebola virus, Congo-Crimean hemorrhagic fever virus, Lassa virus, Machupo virus, Nipah virus, hantavirus, as well as other viruses known in the art.

Rickettsiae are obligate intracellular bacteria that are intermediate in size between most bacteria and viruses and possess certain characteristics common to both bacteria and viruses. Like bacteria, they have metabolic enzymes and cell membranes, use oxygen, and are susceptible to broad-spectrum antibiotics, but like viruses, they grow only in living cells. Although most rickettsiae can be spread only through the bite of infected insects and are not spread through human contact, Coxiella burnetii can infect through inhalation. Examples of rickettsiae that can be harmful to humans are, for example, Rickettsia prowazkeii, Coxiella burnetii, Rickettsia rickettsii, as well as others known in the art.

Fungi are single-celled or multicellular organisms that can either be opportunistic pathogens that cause infections in immunocompromised persons (i.e., cancer patients, transplant recipients, and persons with AIDS) or pathogens that cause infections in healthy persons. Examples of types of fungi that can be harmful to humans are, for example, Blastomyces dermatitidis, Aspergillus, Candida albicans, Coccidioides immitis, Histoplasma capsulatum, Cryptococcus neoformans, Mucorales, Paracoccidioides brasiliensis.

Protozoa are unicellular eukaryotic organisms that feed by ingesting particulate or macromolecular materials, often by phagocytosis. Most protozoa are motile by means of flagella, cilia or amoeboid motion. Examples of protozoan that can be harmful to humans are, for example, Cryptosporidium parvum, Cyclospora cayatanensis, Giardia lamblia, Entamoeba histolytica, Toxoplasma, Microsporidia, Trypanosoma brucei gambiense Trypanosoma brucei rhodesiense, Plasmodium vivax, Plasmodium malariae, Plasmodium ovale, Plasmodium falciparum, as well as others known in the art.

A prion is a protein particle that is capable of causing an infection or disease. Like viruses, prions are not capable of reproduction by themselves, but unlike viruses, prions do not contain genetic material (DNA or RNA). Further, prions have the uncanny ability to change their shape and cause a chain reaction that makes other proteins of the same type change their shape as well. Prions are known to cause a group of devastating neurological diseases known as transmissible spongiform encephalopathies (TSEs), such as, for example, Creutzfeldt-Jakob disease in humans, scrapie in sheep, or bovine spongiform encephalitis in domestic cattle, as well as others known in the art.

In an immunoassay, the phrase “specifically binds to an analyte” or “specifically immunoreactive with,” when referring to an antibody refers to a binding reaction which is determinative of the presence of the analyte in the presence of a heterogeneous population of molecules such as proteins and other biologics (i.e., such as may be found in finished water). Thus, under designated immunoassay conditions, the specified antibodies bind to a particular analyte and do not bind in a significant amount to other analytes present in the sample. A variety of immunoassay formats may be used to select antibodies specifically immunoreactive with a particular analyte. For example, solid-phase ELISA immunoassays are routinely used to select monoclonal antibodies specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity.

Various techniques can be used for transduction including, for example, electro-chemiluminescence, luminescence, fluorescence, surface plasmon resonance and variants, flow cytometry, electrochemistry, and polymerase chain reaction (PCR), with emerging efforts in other optical methods, microcapillary electrophoresis and array technologies. The method of transduction often includes a detectable label. The label may include, but is not limited to, a chromophore, an antibody, an antigen, an enzyme, an enzyme reactive compound whose cleavage product is detectable, rhodamine or rhodamine derivative, biotin, streptavidin, a fluorescent compound, a chemiluminscent compound, derivatives and/or combinations of these markers. Providing a signal with any label is carried out under conditions for obtaining optimal detection of the molecular recognition element. Assays, in particular immunoassays, that utilize particulate moieties as detectable labels are well known to those of skill in the art. Such assays include, but are not limited to fluid or gel precipitin reactions, agglutination assays, immunodiffusion (single or double), immunoelectrophoresis, immunosorbent assays, various solid phase assays, immunochromatography (e.g., lateral flow immunochromatography) and the like. Method of performing such assays are well known to those of skill in the art (see, e.g., U.S. Pat. Nos. 4,366,241; 4,376,110; 4,517,288; 4,837,168; 5,405,784; 5,534,441; 5,500,187; 5,489,537; 5,413,913; 5,209,904; 5,188,968; 4,921,787; and 5,120,643; British Patent GB 2204398A; European patent EP 0323605 B1; Methods in Cell Biology Volume 37: Antibodies in Cell Biology, Asai, ed. Academic Press, Inc. New York (1993); and Basic and Clinical Immunology 7th Edition, Stites & Terr, eds. (1991)).

The methods of this invention are practicable with essentially any assay that uses a particulate moiety as a detectable label. The term particulate moiety is used to refer to any element or compound that is insoluble in the particular buffer system of the immunoassay in which it is utilized or which precipitates out of solution to form a detectable moiety. Typically particulate labels are detected (i.e., recognized as producing a “signal”) when they accrete, agglutinate, or precipitate out of solution to form a detectable mass (distinguishable from the non-accreted, agglutinated or solubilized form of the “particle”), and in one embodiment in a discrete region of the assay medium. Microparticles or “microparticulate labels” are particles or labels ranging in size from about 0.1 nm (average diameter) to about 1000 nm, in one embodiment from about 1 nm to about 1000 nm, in another embodiment from about 10 nm to about 100 nm, and in yet another embodiment from about 15 nm to about 25 nm. In one embodiment, particulate labels include, but are not limited to, particles such as charcoal, kolinite, bentonite, red blood cells (RBCs), colloidal gold, clear or colored glass or plastic (e.g. polystyrene, polypropylene, latex, etc.) beads or microspheres.

Many transduction techniques involve amplification, by either amplifying the signal directly, such as, for example using an enzyme. An enzyme can be used to convert a non-active substrate into an active signal. Further, the use of enzyme amplification can make an assay extremely sensitive because each enzyme molecule can catalyze the production of thousands of product molecules. It is generally the product molecules that are being detected, and thus, large amplification of the output signal can be provided, which enables extraordinarily low levels of detection to be achieved for the targeted agent. For the above reasons, enzymes are commonly used as catalytic labels in transduction of a signal, but in principle any catalytic material can be used, such as an inorganic coordination compound. Alternatively, the target can be amplified, for example, using the polymerase chain reaction (PCR) for nucleic acid, which reduces the sensitivity demanded of the assay by increasing the effective concentration of the target.

In the assay techniques disclosed herein, a molecular recognition element functions to identify a unique component of a targeted biological agent and capture it. The molecular recognition element can be introduced to a sample suspected of having a targeted biological agent (testing sample) using any method known in the art. For example, the molecular recognition elements can be fixed to a solid phase that is non-moveable such as, for example, microwells, capillaries, cuvettes, beads, fibers, as well as others known in the art. In such a case, a testing sample can be introduced to a solid phase that has attached recognition elements. The target biological agent, if present, will be captured and held by the molecular recognition elements fixed on the non-moveable solid phase. Transduction of the captured agent into a signal can be completed while the molecular recognition elements are still fixed to the non-mobile solid phase. Such transduction will be discussed below.

Alternatively, the molecular recognition element(s) can be attached to a mobile solid phase, such as, for example, macro-, micro-, or nanobeads, dipstick, or other moveable solid phase known in the art on which an immunoassay can be performed. For example, at least one molecular recognition element attached to a moveable solid phase can be introduced into a testing sample. Alternatively, a testing sample can be introduced into a solution having at least one mobile solid phase with an attached molecular recognition element. If present, the targeted biological agent will be captured and held by the molecular recognition elements that are attached to the mobile solid phase. Once the targeted biological agents are captured, the final aspect of the immunoassay, transduction can occur.

Using a small, mobile solid phase such as microbeads is advantageous because their size allows them to be dispersed throughout a small testing sample to provide a large surface area to sample volume ratio that enhances the capture of the targeted biological agent by minimizing diffusional distances. Further, the microbeads can be used in small volumes, which reduces the dilution of the signal-providing product in the transduction and detection steps, and therefore, maximizes sensitivity.

The mobile solid phase component may further be magnetic, such as, for example, magnetic nano- or microbeads, which allow the mobile solid phase to be held and/or manipulated by magnets during an assay. In particular, magnetic nano- or microbeads permit the use of a microfluidic assay system where all of the steps can be automated to give near-continuous monitoring. The beads can be transported through channels by fluid flow, captured, and held at specific points by a magnet. An example of a magnetic microbead that can be used is, for example, the 2.8 micron diameter Streptavidin-coated M-280 Dynabeads from Dynal Biotech, Inc. in Great Neck, N.Y.

As previously mentioned, once a molecular recognition element is attached to a solid phase and a targeted biological agent has been identified and captured, either the captured biological agent, or its associated molecular recognition element can be manipulated so that a visible and/or quantifiable signal is present. For example, a signal can be provided by associating the previously captured biological agent with a secondary molecular recognition element that has an attached label, which can be manipulated to emit a signal. Once the secondary molecular recognition element captures the targeted agent, either the label can be manipulated to emit a quantifiable signal, or the label can act to manipulate an added constituent to cause the emission of signal. As previously mentioned herein, such manipulation can occur using, for example, an enzyme. An enzyme, for example, can be attached to a molecular recognition element as a label and react with an enzyme substrate to form an enzyme product that emits a signal. Alternatively, an enzyme substrate attached to a molecular recognition element can be manipulated by an enzyme to form an enzyme product that emits a signal. Alternatively, non-enzyme labels can be used to provide a signal, such as, for example, quantum dots, fluorophores, electrochemical labels, spin, chelated metal labels, liposome labels, radioactive labels, as well as others known in the art. Furthermore, the capture of a targeted agent can be detected without a label using methods such as surface plasmon resonance, scanning microscopies, microcantilevers, as well as other methods known in the art.

Many techniques can be used to detect a signal indicating the presence of a targeted agent. Of these, electrochemistry is an effective detection method when a recognition element is tagged with, for example, an electroactive metal label, an electroactive organic group, or an enzyme that generates an electroactive product. As used herein, electroactive product, electroactive metal label, or electroactive organic groups, refers to those products, metal labels, or organic groups that can be oxidized by the removal of electrons or reduced by the addition of electrons. Electrochemical detection involves an electrochemical cell consisting of at least two electrodes: a working electrode made of a conductive material, such as platinum, gold, or carbon; and a reference electrode, such as a silver wire coated with silver chloride or a saturated calomel electrode. A third electrode, an auxiliary or counter electrode, which is made from a conductive material (i.e., carbon or stainless steel), can also be used. For voltammetric detection, a potential is applied to the working electrode with respect to the reference electrode, and the resulting current is measured. Current arises from the direct transfer of electrons across the electrode/solution interface upon oxidation or reduction of an electroactive species. Electrochemical detection may further include the use of potentiometry, in which the potential between an indicating electrode and the reference is electrode is measured. Thus, the signal indicates the potential of the cell rather than the current. In such a case, the label or enzyme product need not be electroactive. Any method known in the art can be used to conduct an electrochemical detection. Some advantages of electrochemical detection include, for example, detection ability in complicated sample matrices, simple instrumentation, low detection limits, and disposable electrochemical cells.

For example, a secondary molecular recognition element can have an attached enzyme label. An enzyme substrate can be added to the sample containing the captured biological agent and enzyme label. The enzyme that is either added to the testing solution or attached to a secondary molecular recognition element will catalytically convert the substrate to an electroactive product. By way of further example, an enzyme label of, for example, beta-galactosidase can be attached to a secondary molecular recognition element that has captured a targeted agent. An enzyme substrate of, for example, p-aminophenylgalactosidase (PAPG) can then be added to the sample converting the enzyme substrate to p-aminophenol (PAP), which can be electrochemically detected by oxidation. Other enzyme label systems that are known in the art to produce electroactive products can also be used, such as, for example, the use of alkaline phosphatase (ALP) as an enzyme label that converts p-aminophenylphosphate (PAPP) to PAP, which is electrochemically detectable. Examples of some enzyme systems that have been used for electrochemical detection are shown in Table 1. Alternatively, non-enzymatic electrochemical labels can be used such as, for example, metal labels, ferrocenyl labels, as well as others known in the art.

Fluorescence detection is also a commonly used technique to determine the presence of a targeted agent. Fluorescence detection is relatively easy when the fluorophore has a strong luminescence, i.e., when the fluorescence quantum yield is close to unity. In cases where the quantum yield is relatively low, the experimental conditions of fluorescence excitation wavelength, the fluorescence yield, solid angle of the detection optics, and efficiency of the detector all play important roles in determining the overall efficiency of the measurement. In general, the fluorescence methodology can be conducted, for example, using an enzyme label similar to those described above for electrochemical detection. Fluorescence detection methods include, but are not limited to, direct detection of enzyme label emitted fluorescence, detection of fluorescence polarization, detection of fluorescence by resonance energy transfer, detection by quenching of fluorescence, as well as others known in the art. For example, after the initial capture of a targeted agent, a secondary molecular recognition element with an attached enzyme label can recognize and capture a previously captured agent. An enzyme substrate can be introduced into the sample of captured biological agents. The enzyme label can then alter the substrate into an enzyme product that is detectable through fluorescence.

In such a case, various enzymes, such as, for example, ALP and beta-galactosidase can be a label on a molecular recognition element. For these two enzymes, there are multiple fluorescent substrates that can be used to provide adequate fluorescence for detection. For example, fluorescein diphosphate (FDP) reacts with ALP and cleaves both phosphate moieties of the non-fluorescent FDP to produce the highly fluorescent fluorescein dye, which is easily excitable in the visible region at 490 nm with fluorescence emission maximum at 514 nm. The fluorescence quantum yield of fluorescein is known to be pH dependent having a high yield at high pH levels makes FDP a desirable labeled alkaline phosphatase substrate. There are, however, alternative fluorescently labeled alkaline phosphatase substrates that are effective including, for example, 7-hydroxy-9H-(1,3-dichloro-9,9-dimethylacridin-2-one)-phosphate (DDAO-phosphate), 4-methylumbelliferylphosphate (MUP), 6,8-difluoro-4-methylumbelliferylphosphate (DiFMUP). Alternatively, beta-galactosidase can, for example, be used as an enzyme label that reacts with various enzyme substrates, including, for example, fluorescein di-beta-D-pyranoside (FDG), 4-methylumbelliferyl-beta-D-pyranoside (MU-gal), Resorufin beta-D-galactopyranoside (Resorufin-gal), DDAO beta-D-galactopyranoside (DDAO-gal), as well as other enzyme substrates known in the art.

### EXAMPLES

### Example 1

Detecting and enumerating fecal coliforms, especially Escherichia coli, as indicators of fecal contamination, are essential for the quality control of supplied and recreational waters. We have developed a sensitive, inexpensive and small volume amperometric detection method for E. coli β-galactosidase by bead-based immunoassay. The technique uses biotin-labeled capture antibodies (Ab) immobilized on paramagnetic microbeads that have been functionalized with streptavidin (bead˜Ab). The bead˜Ab conjugate captures E. coli from solution. The captured E. coli is incubated in Luria Bertani (LB) broth medium with the added inducer isopropyl β-D-thiogalactopyranoside (IPTG). The induced β-galactosidase converts p-aminophenyl β-D-galactopyranoside (PAPG) into the reduced form of p-aminophenol (PAP), which is measured by amperometry using a 3 mm Au rotating disc electrode. A good linear correlation (R²=0.989) was obtained between log cfu/ml E. coli and the time necessary for the production of a specific amount of PAP. Amperometric detection enabled the determination of 2×10⁶ cfu/mL of E. coli within a 30 min incubation period, and the total analysis time was less than 1 h. It was also possible to determine as few as 20 cfu/mL of E. coli with optimized conditions within 6-7 h. This process may be easily adapted as an automated portable bioanalytical device for the rapid detection of E. coli.

All chemicals were reagent grade and used as received. Aqueous solutions were prepared using organic-pure deionized (DI) water from a Barnstead filtration system (Boston, Mass.). The streptavidin-coated M280 paramagnetic beads (˜2.8 μm) came from Dynal Inc. (Great Neck, N.Y., USA), and were provided as a mono-dispersed suspension of 6.7×10⁸ beads/mL that was used without further dilution. Biotin-conjugated goat Ab to E. coli was purchased from Virostat, Portland, Me. E. coli strain W 3011 was a gift from Professor Brian Kinkle of the Department of Biological Sciences, University of Cincinnati, Ohio, USA. p-Aminophenyl β-D-galactopyranoside, isopropyl β-D-thiogalactopyranoside, and p-aminophenol were from Sigma Chemicals.

The hydrophobic electrochemical cell was constructed as described previously [7]. Briefly, glass slides were wrapped with Parafilm™ (American Can Co., Greenwich, Conn., USA) without stretching or touching the surface to be exposed to the PAPG and the microbeads. The wrapped glass slide was mounted on Plexiglas (3 in×5 in) that also supported Pt wire and Ag wire electrodes.

Three buffers were used. a) 0.1 M PBS, pH 7.5 (250 mL DI water, 1.50 g KH₂PO₄, 2.44 g K₂HPO₄, 1.46 g NaCl; pH adjusted with 6 M HCl or 6 M NaOH); b) PBST, pH 7.5 (0.1M PBS, pH 7.5 with 0.05% v/v Tween 20); c) PBS-D, pH 7.3 (250 mL DI water, 1.50 g KH₂PO₄, 2.44 g K₂HPO₄, 1.46 g NaCl, 0.25 g MgCl₂; pH adjusted with 6 M HCl or 6M NaOH).

Before each experiment, a colony of E. coli, grown on a nutrient agar plate at 37° C. for 24 h, was transferred into Luria Bertani (LB) broth medium and the culture was incubated at 37° C. for 24 h. After incubation the culture was kept at 2-4° C. for 12 h. In all experiments the refrigerated, stored cells were used, and each day a new culture was prepared in LB.

Biotin-conjugated polyclonal Ab against E. coli was coated on the surface of streptavidin-coated paramagnetic beads. In this procedure the initial Ab concentration and incubation time were investigated. The Ab concentration was tested between 0.01 and 0.25 mg/mL. The Ab solutions were prepared from a stock solution (5 mg/mL) and PBS. Streptavidin-coated paramagnetic beads (10 μL) were mixed with 10 μL Ab solution in a capped disposable culture tube and incubated at room temperature for 30 min on a vortex mixer set at the minimum speed. After incubation the beads were removed magnetically and washed (3 times) with 50 μL PBS followed by 50 μL PBST (3 times) and finally with 50 μL PBS (3 times). Ab-coated beads were mixed with 250 μL E. coli (10⁶ cfu/ml) and incubated for 1 h at room temperature on a vortex mixer. After that, the beads were separated magnetically and washed with PBS and PBST. IPTG was mixed with LB broth to prepare the growth medium. The final concentration of IPTG in LB was 0.1 mM, and this was used for inducing β-galactosidase activity. Incubation was at 37° C. for 1 h.

After incubation the growth medium was removed and the beads were washed with PBS. Five μL of beads were then used in the electrochemical measurement.

The effect of incubation time on Ab coating was investigated. The Ab solution (0.15 mg/mL) was incubated with 10 μL streptavidin-coated paramagnetic beads for between 5 and 30 min. After incubation, the beads were removed, washed, exposed to E. coli, and finally the β-galactosidase activity in the cells was determined using the procedure given above.

The effect of the incubation time on capturing E. coli was investigated with the Ab-coated magnetic beads. The beads (Ab concentration: 0.15 mg/mL and incubation time: 10 min) were incubated with E. coli (10⁶ cfu/ml) for between 5 and 40 min at room temperature. After incubation, the beads were removed and washed with PBS and PBST. The beads were incubated in IPTG solution and the β-galactosidase activity in the cells was determined electrochemically.

The effects of other incubation parameters such as IPTG concentration and incubation temperature were also investigated. The effect of the concentration of IPTG in LB was tested between 0 and 0.5 mM using bead-captured E. coli. The E. coli-beads were mixed into 30 μL of LB solution and incubated at 37° C. for 1 h. Then the beads were removed and washed with PBS, and the enzyme activity was determined electrochemically. The effect of the incubation temperature on the growth of E. coli was also examined at 37° C. and 44° C., since these temperatures are frequently used for incubating E. coli. The IPTG concentration in LB was adjusted to 0.5 mM and the E. coli-capture beads were incubated for 1 h. The resulting β-galactosidase activity was determined electrochemically.

We used six E. coli concentrations between 2×10 cfu/mL and 2×10⁶ cfu/mL. A 250 μL aliquot of E. coli solution was mixed with 10 μL of the Ab-coated paramagnetic beads and incubated at room temperature for 20 min while being vortexed. After capturing, the beads were separated magnetically and washed with PBS and PBST. Then, 30 μL of growth solution (prepared by mixing LB with PAPG and IPTG with a final concentration of 4 mM PAPG and 0.5 mM IPTG) were added and incubated at 37° C. For each bacterial concentration, at least four batches of E. coli-beads were prepared. Samples were withdrawn at 30-60 min intervals depending on the E. coli concentration, and the beads were removed from the growth medium. Finally, 5 μL of the growth medium were assayed electrochemically.

The number of cfu/mL in each solution was estimated by serial dilutions spread on MacConkey agar 9 cm Petri dishes. After incubating at 37° C. for 24 h, the number of pink cfus was counted. At least three measurements were made and the average was taken.

Electrochemical measurements were done with a BAS-100B potentiostat and BAS-100W electrochemical software (Bioanalytical Systems, West Lafayette, Ind., USA), with a 3 mm diameter gold rotating disk electrode (RDE), a Pt wire electrode, and a Ag wire reference electrode. A 20 μL drop of 4 mM PAPG in PBS was placed on the surface of the assay platform and the RDE was positioned on the droplet without touching the Pt and Ag electrodes. The BAS was set on Single Potential Time Technique at +290 mV, 100 ms sample interval and 70 s sampling at 2000 rpm. After 40 s had elapsed, 5 μL of sample were added to the PAPG drop. The PAP generated from the enzymatic consumption of PAPG was detected by electrochemical oxidation.

In the later part of the study, the enzymatic hydrolysis of PAPG was done during the incubation period. For that reason, 20 μL of PBS were also used with the PAPG solution on the assay platform surface. After 40 s, 5 μL of PAP sample were added and the oxidation current at the RDE was measured without enzymatic reaction.

Initial studies to evaluate the feasibility of using paramagnetic microbeads to detect bacteria used 30 μL of beads and 30 μL of Ab. Both quantities were reduced to 10 μL to conserve the expensive reagents without affecting the results (data not shown), and therefore the entire assay for this study was done using 10 μL each of beads and Ab.

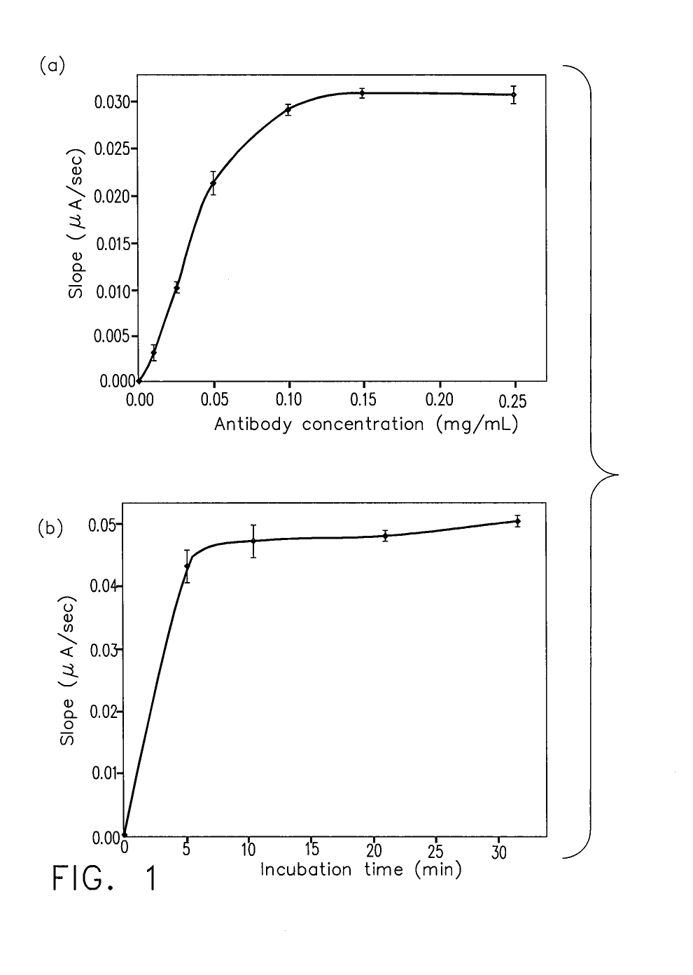

The optimum Ab concentration on the surface of the beads is the minimum amount that yields the maximum response from the maximum concentration of E. coli to be tested. For this determination we exposed a fixed number of beads to Ab concentrations between 0.01 and 0.25 mg/mL, followed by incubation with E. coli. The results are given in FIG. 1(a), where the onset of the plateau shows that the optimal Ab concentration as defined is 0.15 mg/mL. We chose to use 0.15 mg/mL to minimize the effects of variability caused by being too close to the onset.

As part of the assay optimization process, the time needed for the beads and Ab to form the bead˜Ab conjugate was studied with incubation times between 0 min and 30 min. The current signals generated beyond 10 min were essentially constant, and so (FIG. 1(b)) 10 min was used as the optimum incubation time to form the beads˜Ab conjugate.

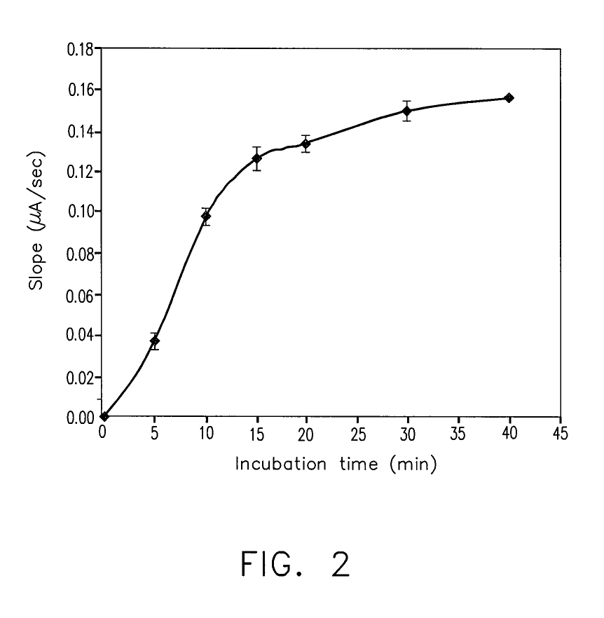

The time involved in capturing E. coli with the bead˜Ab conjugate was also studied with results as shown in FIG. 2. The minimum time to bind E. coli was determined by treating the Ab-beads with 250 μL of a solution of E. coli in LB with capture times from 5 to 40 min. It was expected that the extent of interaction would increase with time and then reach saturation once all available Ab binding sites were occupied. The current signal in amperometric detection increased with increasing capture time up to 20 min, indicating that the available Ab sites were almost saturated. As the current changed by only +15% at 40 min, a 20 min E. coli capture time was used in the subsequent development of the assay.

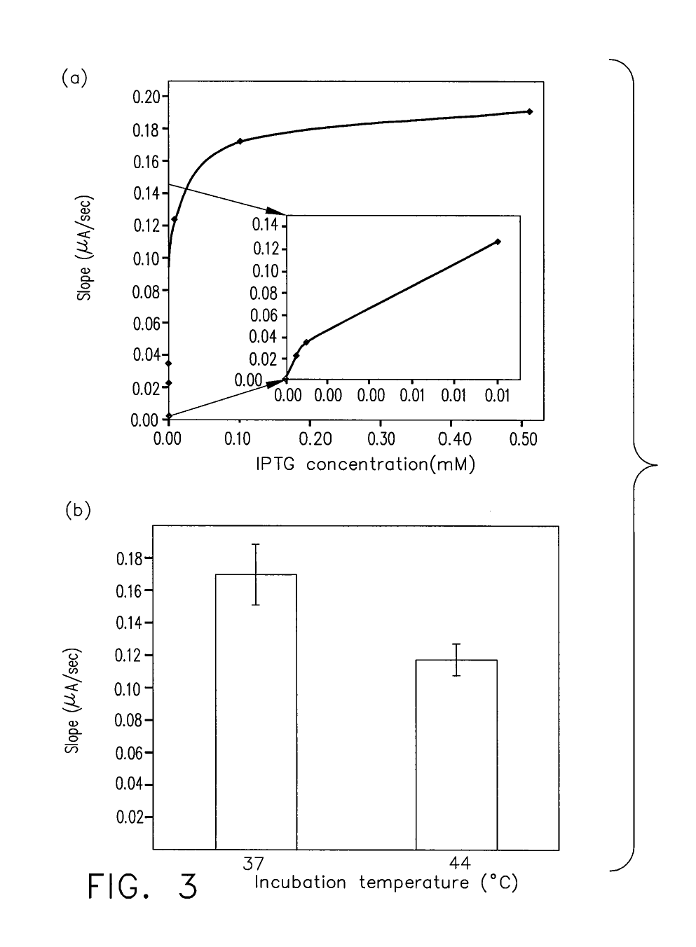

Different IPTG concentrations were used to induce β-galactosidase, which was assayed to determine the optimum IPTG concentration (FIG. 3(a)). IPTG concentrations in LB broth between 0 to 0.5 mM were used. At 0.5 mM IPTG the signal was leveling off, and this concentration was used for induction.

The effect of incubation temperature was investigated at 37° C. and 44° C. (FIG. 3(b)). The signal slope was higher at 37° C., and this was used as the bacterial incubation temperature for the entire study.

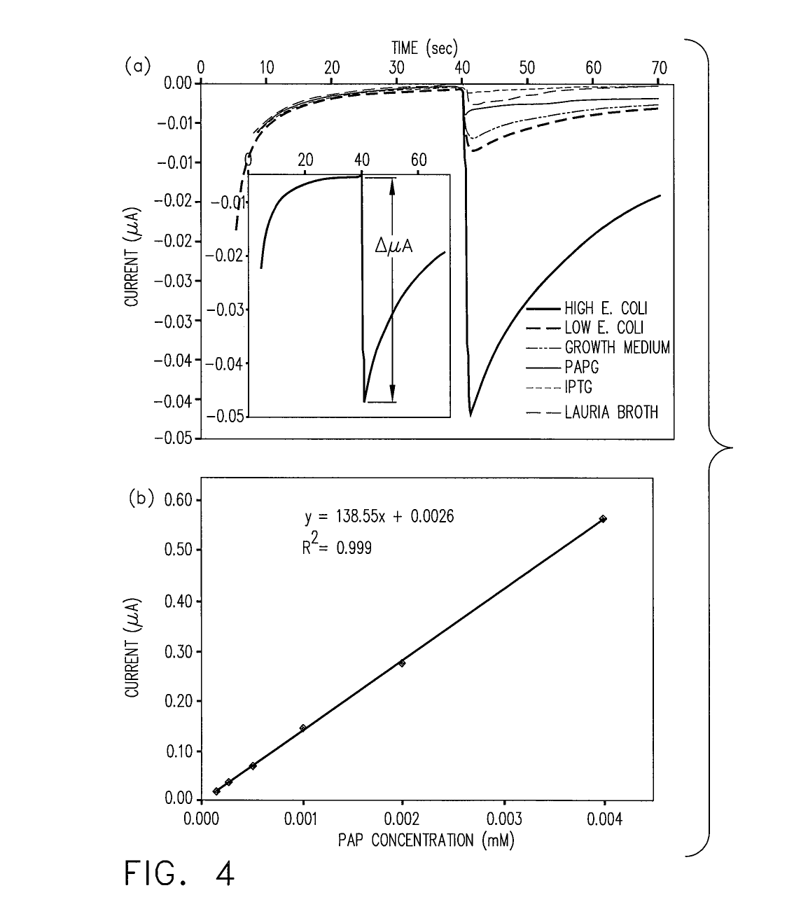

We started by incubating an E. coli cell culture of 10⁶ cfu/mL with 0.5 mM IPTG in LB broth. The production of β-galactosidase was measured electrochemically by following the enzymatic activity using PAPG as substrate. Samples of the bacteria, appropriately diluted, were placed into electrochemical cells. FIG. 4 (a) shows an example of the amperometric response of E. coli cultures of high (10⁶ cfu/mL) and low concentration (10³ cfu/mL) as well as of the blank growth medium. The change of current is related to the enzymatic activity, which is directly related to the concentration of E. coli in the sample solutions.

The performance of the microbial immunoassay was evaluated by generating a calibration curve. The current signal measured amperometrically by RDE was plotted against the concentration of PAP to get the calibration curve shown in FIG. 4(b). A linear relationship between the concentration of PAP and current was obtained (n=6; slope: 139 μA (mmol)⁻¹ PAP; intercept: 0.0026 μA; R²: 0.999).

The detection limit of this immunoassay was calculated from FIG. 4 (b) using the relationship [32]: DL=k S_{bk}/m where DL is the current (nA) at the detection limit, k is a confidence factor, usually 3 for 95% confidence limit, S_{bk} is the standard deviation of the blank measurements (only growth medium) and m is the slope of the calibration curve. The detection limit was 1.0×10⁻⁵ mM PAP, which is better than that of a similar type of assay [33].

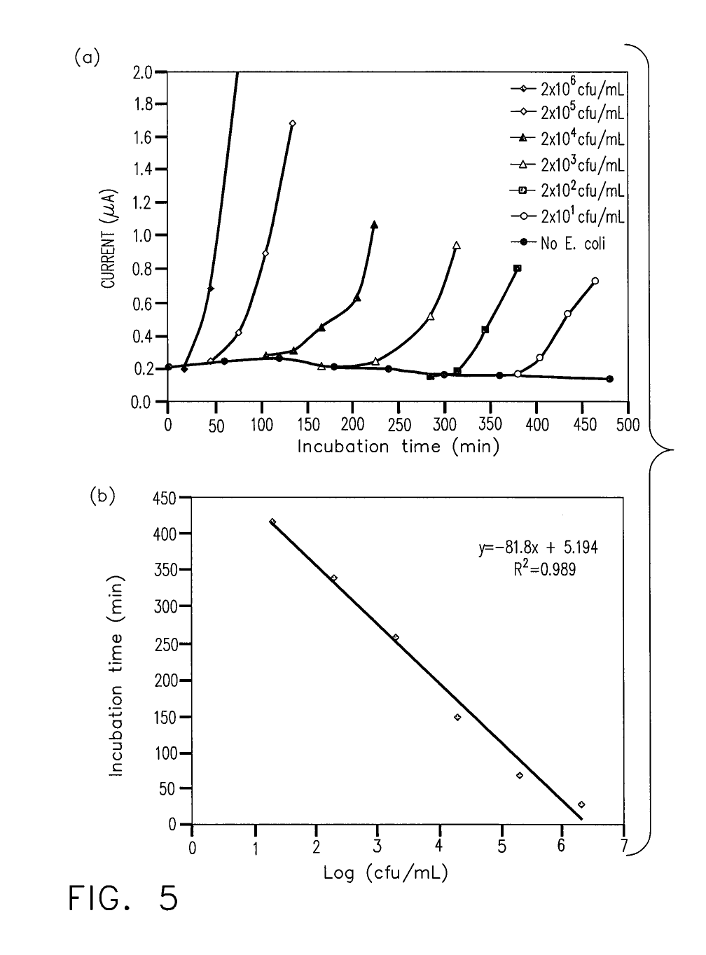

Results obtained for E. coli are shown in FIG. 5 (a). Bacterial concentrations of 2×10⁶ to 20 cfu/mL were used. In the initial flat region, no or very low signals were observed (i.e., <0.34 nA which was three times the standard deviation of the blank). Thus, the number and enzyme activity of the bacteria were insufficient to produce a concentration of PAP equal to or greater than the detection limit. When the bacteria reached a critical concentration, a signal greater than or equal to 0.34 nA was obtained, which then increased rapidly. The time required to obtain a signal of 0.34 nA is reported as the detection time [33].

A semi logarithmic plot of detection time versus initial concentration of E. coli has a good linear correlation with an R² value of 0.989 (FIG. 5(b), and indicates that E. coli at 2×10⁶ cfu/mL could be determined with a 30 min incubation period. Below 20 cfu/mL, it is necessary to incubate the sample for about 7 hours to receive a sensible signal.

### Example 2

A rapid and convenient assay system was developed to detect viable Escherichia coli in water. The target bacteria were recovered from solution by immunomagnetic separation and incubated in tryptic soy broth (TSB) with isopropyl-β-D-thiogalactopyranoside (IPTG), which induces β-galactosidase. Lysozyme was used to lyse E. coli cells and release the β-galactosidase. β-galactosidase converted 4-methylumbelliferyl-β-D-galactoside (MUG) to 4-methylumbelliferone (4-MU), which was measured by fluorescence spectrophotometer using excitation and emission wavelengths of 355 and 460 nm, respectively. The activities of the released enzymes were calculated using calibration graph of 4-MU fluorescence intensities, and a good linear correlation (R²=0.99) was obtained between log cfu mL⁻¹ and log β-galactosidase activity. Detection and enumeration of E. coli was demonstrated with a detection range of 4×10¹ to 4×10⁶ cfu mL⁻¹ and an incubation time of 120 min. The developed immunoassay did not require enrichment or filtration and needed only one antibody, which makes the assay less expensive. Direct detection of viable cells can be done by the method, since it was based on the activity of the enzyme intrinsic to E. coli.

Streptavidin-coated M280 paramagnetic beads of 2.8 μm in diameter, were from Dynal Inc. (Great Neck, N.Y., USA) as a mono-dispersed suspension of 6.7×10⁸ beads ml⁻1 were. Biotin-conjugated goat antibody to E. coli was from Virostat, Portland, Me. E. coli K12 strain was from Refik Saydam National Type Culture Collections, Ankara, Turkey. Sorbitol MacConkey agar (SMAC) and tryptic soy broth (TSB) were from Merck KGaA (Germany). 4-methylumbelliferyl-β-D-galactoside (MUG), 4-methylumbelliferone (4-MU), dimethylsulfoxide, isopropyl-β-D-thiogalactopyranoside (IPTG) were from Sigma Chemicals Co. (St. Louis, Mo.). Na₂HPO₄ and KH₂PO₄ were from J. T. Baker (Netherlands), used as phosphate buffer saline (PBS).

Fluorescence measurements were done using a Cary Eclipse Fluorescence Spectrophotometer (Varian Inc., Netherlands) using excitation and emission wavelengths of 355 and 460 nm, respectively, and a quartz micro cell (50 μL). The temperature was controlled by Cary Eclipse software and a Peltier system.

The streptavidin-coated paramagnetic beads (10 μl, 6.7×10⁸ beads ml⁻1) were added to a tube containing biotin-conjugated antibodies (Ab) (10 μl, 0.15 mg ml⁻1), and the tube was vortexed (Stuart, UK) at room temperature for 10 min [8]. The Ab-coated beads were removed magnetically from the solution and washed 3 times by resuspending them in PBS (pH 7.5, 0.1 M). The beads were then mixed with E. coli (2-4×10³ cfu ml⁻1) and incubated at room temperature on the vortex mixer for various times from 0-60 min to determine the effect of reaction time on capturing E. coli. Similarly, the effects on capturing of PBS concentration and pH, and the reaction volume were examined. The bead-E. coli complexes were manipulated magnetically. A 100 μl sample solution containing E. coli, and 100 μl supernate solution containing uncaptured E. coli were plated on SMAC and incubated at 37° C. for 24 h. E. coli colonies were counted to determine the percentage of the captured E. coli. The rest of the supernate was removed and the beads were washed 3 times with PBST (pH 7.5, 0.1 M, 0.05% (v/v) Tween 20). IPTG (40 μl, 0.5 mM), dissolved in TSB, was mixed with the captured E. coli to induce β-galactosidase activity, and incubated at 37° C. for 2 h [8].

Lysozyme (10 μl, from 0-20 mg mL⁻1) was added to captured E. Coli (10⁶ cfu mL⁻1) to lyse the cells to release the β-galactosidase, and incubated at 37° C. for 0-45 min. Measurement was done in a 50 μL final volume, consisting of 20 μL PBS (0.1 M, 1 mg mL⁻1 MgCl2), 10 μL MUG solution and 20 μL sample. MUG solution was prepared with dimethylsulfoxide-PBS buffer and used as the substrate for β-galactosidase. The activities of the released enzymes were calculated using calibration graphs of 4-MU fluorescence intensities which were derived for the same pH and temperature.

E. coli solution (10⁶ cfu mL⁻1), in which the β-galactosidase activity had been induced with IPTG (0.5 mM), was incubated with lysozyme (5 mg mL⁻1) at 37° C. for 30 min. This solution was used as a stock enzyme solution. Measurement was done in 50 μL final volume of 20 μL PBS (0.1 M, 1 mg mL⁻1 MgCl2), 10 μL MUG solution and 20 μL enzyme solution. The effect of temperature on enzyme activity was investigated between 22 and 67° C., (at pH 7.3 and 0.5 mM MUG) and the effect of pH was investigated between 6.5 and 7.7 at 37° C. and 0.5 mM MUG. The effect of MUG concentration on enzyme activity was investigated similarly, at 37° C. and pH 7.3 while varying the MUG concentration in the reaction medium between 0.05 to 2 mM. The activity of the enzyme was calculated using calibration graphs of 4-MU fluorescence intensities which were derived for different pH and temperatures. E. coli (10¹ cfu mL⁻1 and 10⁶ cfu mL⁻1) were captured by magnetic beads and lysed after the induction of the β-galactosidase activity. Sample (20 μL) was added to a quartz micro cell containing 20 μL PBS (pH 7.3, 0.1 M, 1 mg mL⁻¹ MgCl₂) and 10 μL MUG (1 mM) at 50° C. The bacterial cell count was detected by measuring the slope of the increase in intensity of 4-MU, the product of the enzyme reaction.

The number of cfu ml⁻1 in each solution was estimated by plating on SMAC, incubating at 37° C. for 24 h, and counting the number of colonies. The average was taken of at least three measurements.

The effect of reaction time from 10-60 min on capturing E. coli (250 μl, 3.0×10³ cfu mL⁻1) by antibody-coated paramagnetic beads at room temperature and pH 7.5, 0.1 M PBS. The percentage of captured bacteria increased with increasing reaction time up to 30 min, reached approximately 60 % and flattened off.

The effect of pH of PBS (0.1 M) on bacteria capturing (250 μl, 3.0×10³ cfu mL⁻1) at room temperature for 30 min is shown in FIG. 2. A maximum efficiency (approximately 60%) of capturing bacteria was observed at pH 7.5.

The effect of PBS concentration on capturing E. coli (250 μl, 3.5×10³ cfu mL⁻1) at room temperature and pH 7.5 for 30 min was determined (Figure not shown). A maximum efficiency (approximately 60%) of capturing bacteria was observed at 0.1 M PBS concentration. One of the most important factors that affect the antibody-antigen interaction is ionic bonds [9]. Thus, the changes in the pH or ionic strengths of the reaction medium can easily affect the binding of antigen to the antibody.

The effect of immunoreaction volume on capturing bacteria was examined from 50 to 500 μL (Fig. not shown). The same amounts of bacteria (7.5×10² cfu) and antibody-coated magnetic beads (6.7×10⁸ beads) were used in different volumes and the capturing was realized at room temperature and pH 7.5 in 0.1 M PBS for 30 min. When the reaction volume reached 250 μL, the number of captured bacteria was a maximum and then decreased with increasing volume. The second part of this graph was expected, in that increasing the immunoreaction volume reduces the density of beads and bacteria; as a result, the probability of interaction between beads and bacteria would be decreased and this change negatively affected the capturing efficiency. On the other hand, the first part of the graph shows an unexpected trend. Decrease in immunoreaction volume, which increased bead and bacteria concentration, dramatically decreased capturing efficiency (capturing efficiency at 50 μl and 250 μl were 30 and 58%, respectively). These data were confirmed by measuring the enzyme activity in the captured cells and a similar change was obtained (data not shown). An increase in bead density may cause steric hindrance on the immunoreaction and this hindrance would affect the efficiency negatively.

The maximum amount of released enzyme was found at 5 mg mL⁻1 lysozyme concentration. Above this concentration, the amount of released enzyme decreased up to 21% with increasing lysozyme concentration. The effect of reaction time on releasing enzyme was determined (Figure not shown). The amount of released enzyme increased with increasing incubation time up to 30 min.

We monitored the activity at various temperatures from 22 to 67° C. to determine the optimal temperature for β-galactosidase activity (Fig. not shown). The maximum activity of the enzyme was observed at 53° C. The optimal enzyme activity was observed at pH 7.25

The effect of substrate concentration on β-galactosidase activity was examined in the presence of various concentrations of MUG (Figure not shown). The enzymatic activity first increased with the increase of MUG concentration up to 1.0 mM, and then decreased with increase of substrate concentration due to the substrate inhibition of β-galactosidase [10].

The optimized parameters of the developed assay were applied to detecting E. coli. The system was used with stock solutions containing concentrations of E. coli between 4×10¹ and 4×10⁶ cfu mL⁻1. A good linear correlation (R²=0.99) was obtained between log cfu mL⁻¹ E. coli and log β-galactosidase activity. The working range was 4×10¹ to 4×10⁶ cfu mL⁻¹ with an incubation time of 120 min. Total analysis time of the measurement, which includes bacteria capturing (30 min), incubation with IPTG (120 min), lysis of the cells (30 min) and fluorescence measurement and other (20 min), was less then 200 min. Using three times the noise, the detection limit of the proposed method is 40 cfu ml⁻¹and if this value is multiplied with capturing efficiency and sample volume, the number of the bacteria captured on the beads is obtained. It is 6 cfu, which was captured by the beads and could be detected by the method. If the incubation period is increased it would not have the effect of reducing the detection limit, since the number of the bacteria in the reaction medium is limited. It is possible to increase the number of captured bacteria by increasing the sample volume. However, if the sample volume is increased, then the amount of the antibody-coated beads should be increased. Otherwise, due to the reduction of bead density in the capture volume, the efficiency of capture will decrease and this change negatively affects the performance of the measurement. Increasing the number of beads will also increase the cost of the measurement. If the E. coli concentration in the sample is higher than 10³ cfu mL⁻¹, a 60 min incubation with 120 min overall analysis time will be enough to detect E. coli. The analysis system is adaptable to an automated fluidic system, which will be investigated in further studies.

### REFERENCES

[1] B. Swaminathan, P. Feng, Rapid detection of food-borne pathogenic bacteria, Ann. Rev. of Microbiol. 48 (1994) 401-406.

[2] D. L. Archer, J. E. Kvenberg, Incidence and cost of foodborne diarrheal disease in United States, J. of Food Protect 48 (1985) 887-894.

[3] T. E. Ford, Microbiological safety of drinking water: United States and global perspectives, Environ. Health Perspect. 107 (1999) 191-206.

[4] W. R. MacKenzie, N. J. Hoxie, M. E. Proctor, M. S. Gradus, K. A. Blair, D. E. Peterson, J. J. Addiss, K. R. Fox, J. B. Rose, J. P. Davis, A massive outbreak in Milwaukee of Cryptosporidium infection transmitted through the public water supply, N. Eng. J. Med. 331 (1994) 161-167.

[5] Centers for Disease Control and Prevention, Morb. Mortal. Wkly. Rep. Outbreak of Escherichia coli O157:H7 and Campylobacter among attendees of the Washington Country Fair-New York 48 (1999) 803-805.

[6] W. Kondro, E. coli outbreak deaths spark judicial inquiry in Canada, Lancet 355 (2000) 2058.

[7] J. H. Thomas, N. J. Ronkainen-Matsuno, S. Farrell, H. B. Halsall, W. R. Heineman, Microdrop analysis of a bead-based immunoassay, Microchemical J. 74 (2003) 267-276.

[8] S. F. Altekruse, M. L. Cohen, D. L. Sherdlow, Emerging foodborne disease, Emerg. Infec. Dis. 3 (1997) 285-293.

[9] P. M. Griffin, R. V. Tauxe, The epidemiology of infections caused by Escherichia coli O157:H7, other enterohemorrhagic E. coli, and the associated hemolytic uremic syndrome, Tauxe, Epidemiol. Rev. 13 (1991) 60-98.

[10] D. L. Jones, Potential health risks associated with the persistence of Escherichia coli O157 in agricultural environments, Soil Use and Management 15 (1999) 76-83.

[11] G. A. Willshaw, J. Thirlwell, A. P. Jones, S. Parry, R. L. Salmon, M. Hickey, Vero cytotoxin-producing Escherichia coli O157 in beefburgers linked to an outbreak of diarrhea, haemorrhagic colitis and haemolytic uraemic syndrome in Britain, Lett. Appl. Microbiol. 19 (1994) 304-307.

[12] E. W. Rice, M. J. Allen, D. J. Brender, S. C. Edberg, Assay for beta-glucuronidase in species of the genus Escherichia and its applications for drinking-water analysis, Appl. Environ. Microbiol. 57 (1991) 592-593.

[13] American Public Health Association, Standard methods for the examination of water and wastewater, 19^{th} edition, American Public Health Association, Washington D.C., (1995) Parts 921 1B, 921 1C, 922 1B and 9222.

[14] A. J. Madonna, S. V. Cuyk, K. J. Voorhees, Detection of Escherichia coli using immunomagnetic separation and bacteriophage amplification coupled with matrix laser desorption/ionization time-of-flight mass spectrometry, Rapid Commun. Mass Spectrom. 17 (2003) 257-263.

[15] K. Geissler, M. Manafi, I. Amoros, J. L. Alonso, Quantitative determination of total coliforms and Escherichia coli in marine waters with chromogenic and fluorogenic media, J. Appl. Microbiol. 88 (2000) 280-285.

[16] M. Hattenberger, F. Mascher, K. Kalcher, E. Marth, Improved method for the fluorimetric detection of β-D-galactosidase in water, Int. J. Hyg. Environ. Health. 203 (2001) 281-287.

[17] G. R. Campbell, J. Prosser, A. Glover, K. Killham, Detection of Escherichia coli O157:H7 in soil and water using multiplex PCR, J. Appl. Microbiol. 91 (2001) 1004-1010.

[18] W. Sun, F. Khosravi, H. Albrechtsen, L. Y. Brovko, M. W. Griffiths, Comparison of ATP and in vivo bioluminescence for assessing the efficiency of immunomagnetic sorbents for live Escherichia coli O157:H7 cells, J. Appl. Microbiol. 92 (2002) 1021-1027.

[19] A. M. Prescott, C. R. Fricker, Use of PNA oligonucleotides for the in situ detection of Escherichia coli in water, Mol. Cell. Probes 13 (1999) 261-268.

[20] T. Hurt, R. D. Craven, K. G. Harvey, P. Zhang, E. Price, N. C. Fawcett, J. A. Evans, Detection of E. coli O157:H7 with a quartz crystal biosensor and asymmetric PCR, Presented at INABIS'98—5^{th} Internet World Congress on Biomedical Sciences at McMaster University, Canada, (1998) December 7-16^{th}.

[21] H. Stender, A. J. Broomer, K. Oliveira, H. Perry-O'Keefe, J. J. Hyldig-Nielsen, A. Sage, J. Coull, Rapid detection, identification, and enumeration of Escherichia coli cells in municipal water by chemiluminescent in situ hybridization, Appl. Environ. Microbiol. 67 (2001) 142-147.

[22] W. C. Yam, M. L. Lung, M. H. Ng, Evaluation and optimization of a latex agglutination assay for detection of cholera toxin and Escherichia coli heat-labile toxin, J. Clin. Microbiol. 30 (1992) 2518-2520.

[23] P. Arbault, V. Buecher, S. Poumerol, M. L. Sorin, Study of ELISA method for detection of E. coli O157 in food, Progress in Biotechnol. 17 (2000) 359-368.

[24] H. Yu, J. G. Bruno, Immunomagnetic-electrochemiluminescent detection of Escherichia coli O157 and Salmonella typhimurium in foods and environmental water samples, Appl. Environ. Microbiol. 62 (1996) 587-592.

[25] J. A. Ho, H. Hsiu-Wen, Procedures for preparing Escherichia coli O157:H7 immunoliposome and its application in liposome immunoassay, Anal. Chem. 75 (2003) 4330-4334.

[26] P. Bouvrette, J. H. T. Luong, Development of a flow injection analysis (FIA) immunosensor for the detection of Escherichia coli, Int. J. Food Microbiol. 27 (1995) 129-137.

[27] C. Ruan, H. Wang, Y. Li, A bienzyme electrochemical biosensor coupled with immunomagnetic separation for rapid detection of Escherichia coli O157:H7 in food samples, Transactions of the ASAE, 45 (2002) 249-255.

[28] T. Neufeld, A. Schwartz-Mittelmann, D. Biran, E. Z. Ron, J. Rishpon, Combined phage typing and amperometric detection of released enzymatic activity for the specific identification and quantification of bacteria, Anal. Chem. 75 (2003) 580-585.

[29] C. A. Wijayawardhana, H. B. Halsall, W. R. Heineman, Micro volume rotating disk electrode (RDE) amperometric detection for a bead-based immunoassay, Anal. Chim. Acta 399 (1999) 3-11.

[30] C. A. Wijayawardhana, G. Wittstock, H. B. Halsall, W. R. Heineman, Electrochemical immunoassay with microscopic immunomagnetic bead domains and scanning electrochemical microscopy, Electroanalysis 12 (2000) 640-644.

[31] S. Purushothama, S. Kradtap, C. A. Wijayawardhana, H. B. Halsall, W. R. Heineman, Small volume bead assay for ovalbumin with electrochemical detection, Analyst 126 (2001) 337-341.

[32] J. D. Jr. Ingle, S. R. Crouch, Spectrochemical Analysis, Prentice Hall, 1988, p 173.

[33] F. Perez, I. Tryland, M. Mascini, L. Fiksdal, Rapid detection of Escherichia coli in water by a culture-based amperometric method, Anal. Chim. Acta 427 (2001) 149-154.

[34] A. S. Mittelmann, E. Z. Ron, J. Rishpon, Amperometric quantification of total coliforms and specific detection of Escherichia coli, Anal. Chem. 74 (2002) 903-907.

We claim:

1. A method of measuring the presence of a live microorganism of interest in a sample, comprising the steps of:
a. capturing the microorganism of interest with an appropriate amount of targeting moiety capable of binding specifically to the target microorganism of interest;
b. incubating the microorganism with a substrate for an enzyme present in the microorganism for a time sufficient to allow production of a detectable amount of product by the enzyme in the live microorganisms present;
c. detecting the product; and
d. correlating the amount of product with a known standard and thereby determining the presence of live microorganisms.

2. The method of claim 1 further comprising the step of obtaining a sample to be tested from a source where contamination is suspected.

3. The method of claim 1 further comprising the step of incubating the microorganism for a time sufficient to allow growth of the live microorganisms present.

4. The method of claim 1 wherein the sample is used to monitor the biological contamination level in drinking water.

5. The method of claim 1, wherein the method further comprises the step of incubating the microorganism with an inducer reagent.

6. The method of claim 5, wherein the inducer reagent includes an inducer compound that induces the activity of an enzyme unique to the microorganism of interest.

7. The method of claim 5, wherein the inducer is isopropylthiogalactopyranoside (IPTG).

8. The method of claim 5, wherein the inducer is selected from the group consisting of 1-O-methyl-beta-D-glucuronide, isopropyl-beta-D-thioglucuronic acid, isopropyl-beta-D-thiogalactopyranoside, 3-O-methyl-alpha-D-glucopyranoside and 1-O-methyl-beta-D-glucopyranoside.

9. The method of claim 1, wherein the substrate comprises an indicator reagent.

10. The method of claim 9, wherein the indicator reagent includes an indicator compound that undergoes a change detectable by spectrophotometric or visual methods upon cleavage by a beta galactosidase enzyme found in coliforms or a beta glucuronidase enzyme unique to E. coli.

11. The method of claim 1 further comprising the step of incubating the test sample and control sample at about 35° C. for about 24 h or less.

12. The method of claim 1 further comprising the step of lysing the cell membranes of the mircroorganism in order to release the enzyme to which the substrate is directed.

13. A kit for rapidly and accurately determining and indicating the presence or absence of viable microorganisms in a sample comprising:
a. a first reagent containing a paramagnetic bead coated with a paratropic agent specific for the target microorganism and capable of forming a complex with the target microorganism;
b. a second reagent separated from said first reagent which contains a substrate suitable for the microorganism to be detected; and
c. a third reagent separated from said first and second reagents which contains a standard for the product produced by the substrate.

14. The kit of claim 13, wherein the substrate is capable of production of a detectable product by the enzyme of interest in the live microorganisms.

15. The kit of claim 13 further comprising an inducer reagent for the enzyme of interest in the microorganism.
